(19) United States
(12) Patent Application Publication
(10) Pub. No.: US 2015/0017201 A1
(43) Pub. Date: Jan. 15, 2015

(54) NOVEL NICOTINE DNA VACCINES

(71) Applicants: Regents of the University of Minnesota, Minneapolis, MN (US); Arizona Board of Regents, A Body Corporate of the State of Arizona, Acting for and on behalf of Ariz, Scottsdale, AZ (US)

(72) Inventors: Yung Chang, Tempe, AZ (US); Hao Yan, Chandler, AZ (US); Sidney Hecht, Phoenix, AZ (US); Paul Pentel, Minneapolis, MN (US); Xiaowei Liu, Tempe, AZ (US); Rumit Maini, Chandler, AZ (US)

(21) Appl. No.: 14/209,845

(22) Filed: Mar. 13, 2014

Related U.S. Application Data
(60) Provisional application No. 61/798,635, filed on Mar. 15, 2013.

(51) Int. Cl. A61K 39/385; A61K 39/39; A61K 39/00

(52) U.S. Cl. CPC A61K 39/385; A61K 39/0013; A61K 39/39
USPC 424/193.1; 536/23.1; 530/403

(57) ABSTRACT

The present invention provides DNA-nanostructures comprising and at least one targeting moiety, wherein the at least one targeting moiety is linked to the DNA-nanostructure; and wherein the at least one targeting moiety is nicotine or a nicotine analogue. These compounds elicit an immunogenic response in individuals and are useful as vaccines for ameliorating nicotine dependence.

### CROSS-REFERENCE TO RELATED APPLICATION

This application claims the benefit under 35 U.S.C. §119(e) of U.S. Provisional Application No. 61/798,635, filed Mar. 15, 2013, the entire contents of which is hereby incorporated by reference.

### STATEMENT REGARDING FEDERALLY SPONSORED RESEARCH

This invention was made with government support under R21DA030045 awarded by the National Institutes of Health and National Institute on Drug Abuse and 5R01DA035554 awarded by the National Institutes of Health. The government has certain rights in the invention.

### BACKGROUND OF THE INVENTION

Cigarette smoking is the most common preventable cause of death, accounting for more than 440,000 fatalities in the United State alone. Despite the obvious health risks smoking is still prevalent (20-25% of American adults), primarily due to nicotine (NIC) dependence. In addition to NIC replacement and behavior intervention, neither of which is very effective, NIC vaccines have been explored as a method to reduce tobacco dependence (1).

Although recent data on NicVax, the most advanced NIC vaccine, are disappointing, there is a clear correlation between a high level of elicited NIC-specific antibodies and a reduction in NIC dependence as observed in both animal studies and clinical trials (2,3). This result demonstrates the therapeutic potential of these vaccines, but also highlights the importance and challenge of improving vaccine efficacy, warranting further research into vaccine design (3).

Many clinically relevant NIC vaccines are synthesized by a protein-hapten conjugation strategy in which hapten/carrier stoichiometry and hapten spacing cannot be controlled (4,5). Thus, protein-hapten conjugates do not facilitate simple systematic evaluation and are difficult to modify for enhanced hapten immunogenicity.

Alternatively, nicotine vaccines have been explored to reduce tobacco dependence, by preventing the nicotine molecules from getting into the brain and promoting dopamine release, however, these vaccines have demonstrated limited efficacy.

Accordingly, new therapeutic strategies and approaches are needed for smoking cessation. In particular, new nicotine vaccines and therapeutics are needed.

### SUMMARY OF THE INVENTION

In an aspect of the present invention, there is provided a compound comprising a DNA-nanostructure and at least one targeting moiety, wherein the at least one targeting moiety is linked to the DNA-nanostructure; and wherein the at least one targeting moiety is nicotine or an analogue thereof.

In another aspect, there are provided compositions comprising the aforementioned compounds in combination with a physiologically-acceptable, non-toxic vehicle.

The present invention provides a method of inducing an immune response in a subject, comprising administering to the subject a therapeutically effective amount of a compound of the invention.

The present invention provides a method of inducing the production of high affinity neutralizing antibodies or inhibitory antibodies comprising administering a compound of the invention to a subject having an addiction to nicotine.

The present invention provides a method of inducing a therapeutic immune response in a subject having an addiction to nicotine, comprising administering to the subject a therapeutically effective amount of a compound of the invention.

The present invention provides a method for treating a subject with an addiction to nicotine comprising administering a therapeutically effective amount of a composition of the invention to the subject.

The present invention provides a method for preventing nicotine addiction in a subject comprising administering a therapeutically effective amount of a composition of the invention to the subject.

In certain embodiments, the subject is a mammal (e.g., a human).

The present invention provides the use of a compound of the invention for the manufacture of a medicament useful for the treatment of addiction to nicotine in a subject.

The present invention provides compounds of the invention for use in therapy.

### BRIEF DESCRIPTION OF THE FIGURES

FIG. 1. Illustration of a 12-arm DNA-nicotine nanostructure incorporating CpG motif and T-helper peptides.

FIG. 2. Illustration of immunization of mice with DNA nanostructures and their internalization upon specific binding to B cells and non-specific binding to dendritic cells and macrophages as well as activation by T cells.

FIG. 3. Surface display of nicotine assembled onto DNA.

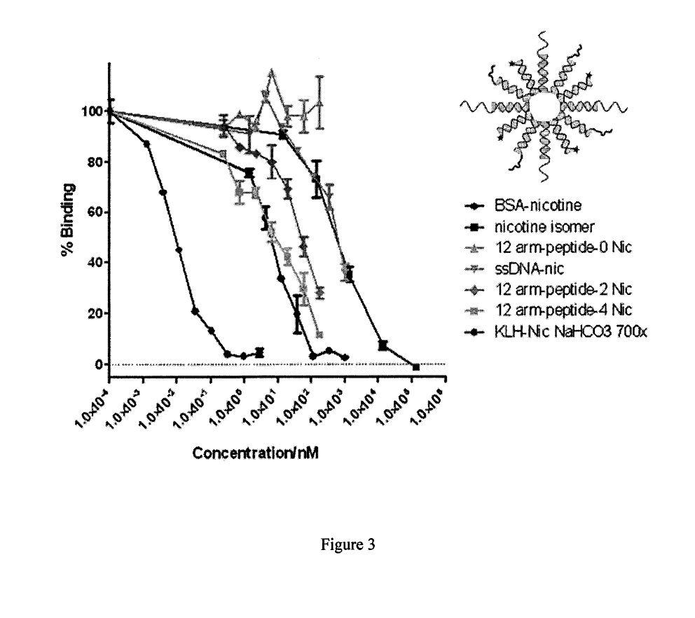

FIG. 4. Antigen internalization in mouse macrophage cells.

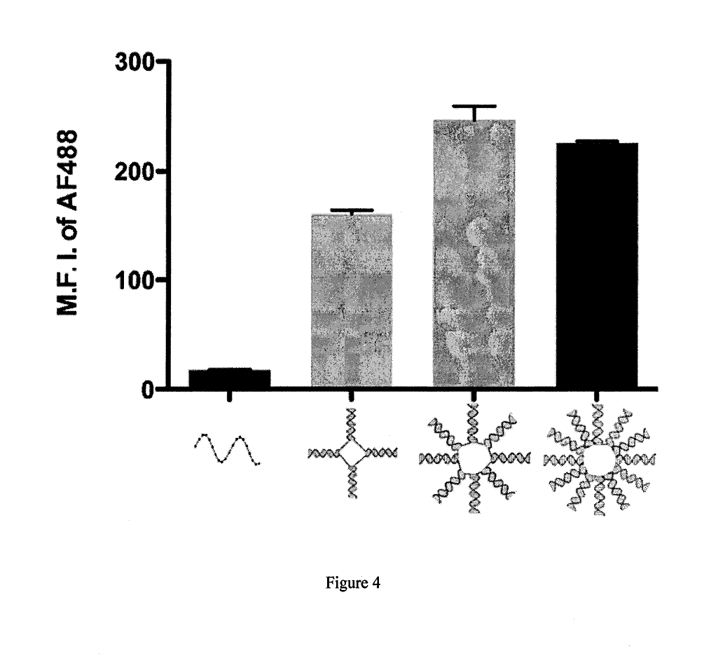

FIG. 5. Anti-nicotine antibody responses. DNA-nicotine nanostructure elicited far greater antibody titer than control.

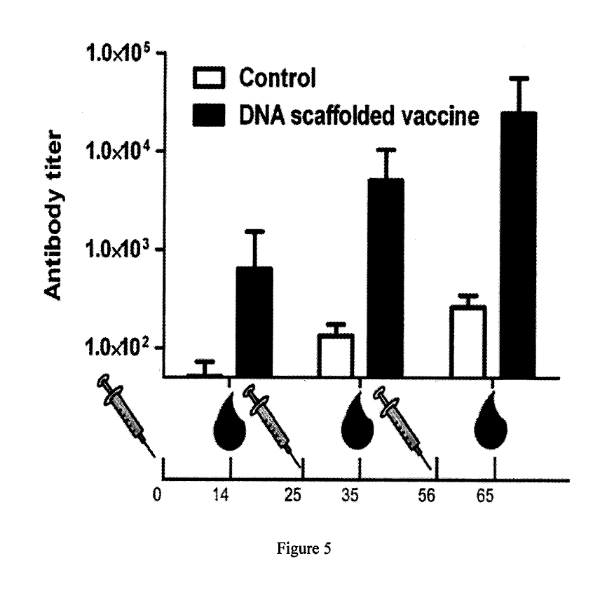

FIG. 6. Examples of organizing proteins and nanoparticles by DNA-directed self-assembly. A) Schematic diagram and AFM image of the programmed self-assembly of streptavidin on a 2D DNA nanogrid. AFM image is 1000 nm². B) Cartoon and AFM image of the aptamer-directed self-assembly of thrombin arrays on DNA templates. Image is 500 nm². C) and D) Single molecule visualization of the bivalent aptamer-protein (thrombin in C and PDGF in D) binding using AFM. In this case, aptamers are displayed in lines on a rectangular shaped DNA tile. The dual aptamer line possesses stronger binding affinity with protein than each individual aptamer lines.

FIG. 7. Competitive inhibition of interactions between fluorescently-labeled, monomeric aptamers by mono- or multi-valent aptamer scaffolds. A. Illustration of various aptamers that are specific to a B cell line. B. Fluorescence intensity assessed by flow cytometry (y-axis) of the cells after incubation with labeled monomer in the presence of various concentrations of unlabeled monomeric, dimeric, or tetrameric aptamers at the indicated concentrations (x-axis). C. The concentrations that cause 50% inhibition of fluorescence intensity, i.e. of IC50 by various aptamer scaffolds.

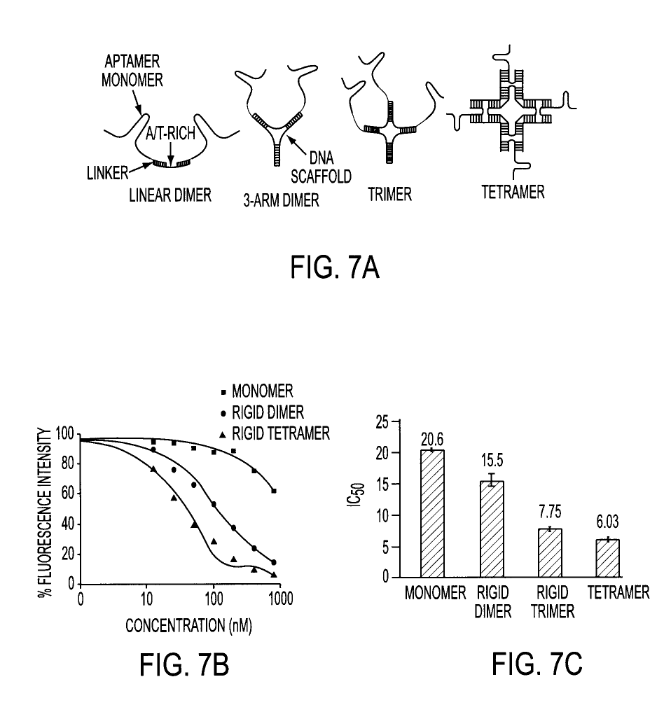

FIG. 8. Size-dependent delivery of DNA nanoarrays into different cellular compartments. Various structures of fluorescence conjugated DNA (shown on the left), such as oligomer (top), tetramer of the same oligomers (middles) or DNA origami (bottom) were incubated with a human cell lines, and their cellular localization was analyzed by fluorescence microscopy (right).

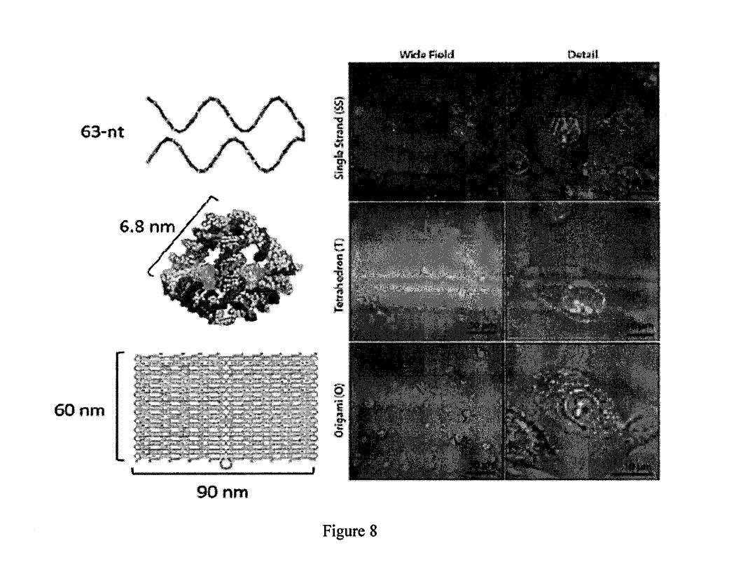

FIG. 9. Various nicotine/adjuvant DNA—origami constructs. A) nicotine (ball) anchored to the DNA-origami through hairpin-DNA (line); B): nicotine linked to peptide (line)-oligonucleotide that is hybridized to DNA-origami; C) nicotine conjugated peptide-oligonucleotide that is hybridized to DNA origami. D) DNA-origami containing TLR ligands, such as dsRNA, CpG-DNA or functionalized TLR-7 ligands.

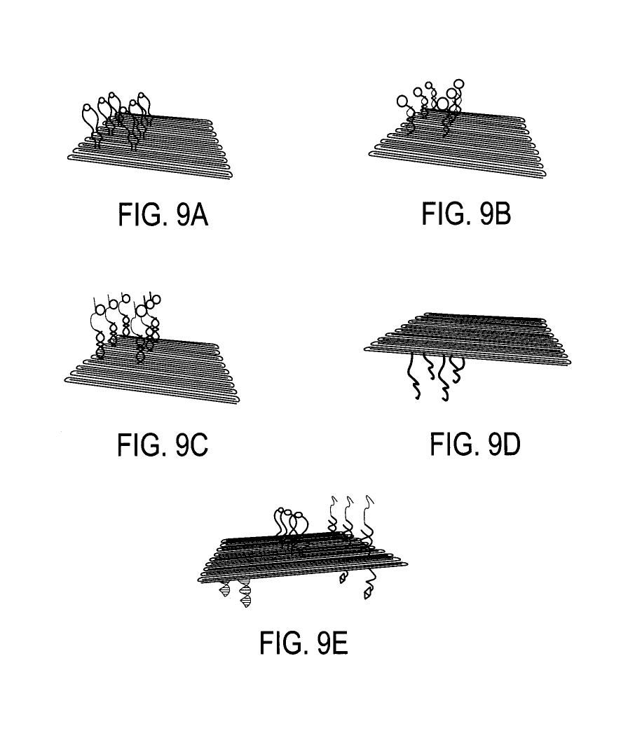

FIG. 10. Scheme for synthesis and activation of a functionalized TLR7/8.

FIG. 11. Some nicotine haptens for DNA nanostructures.

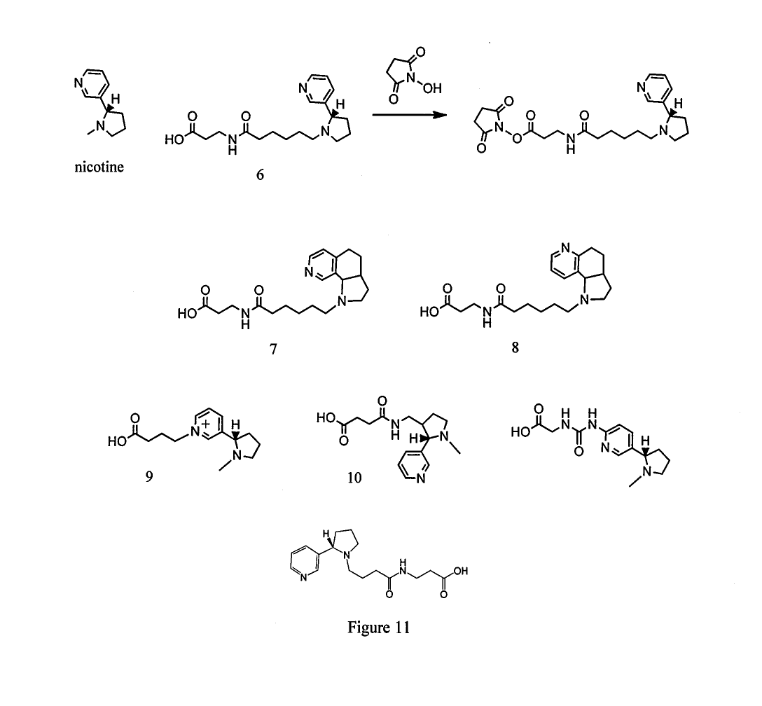

FIG. 12. Synthesis of a thymidine phosphoramidite conjugated to a nicotine hapten through the 5-position of the nucleobase.

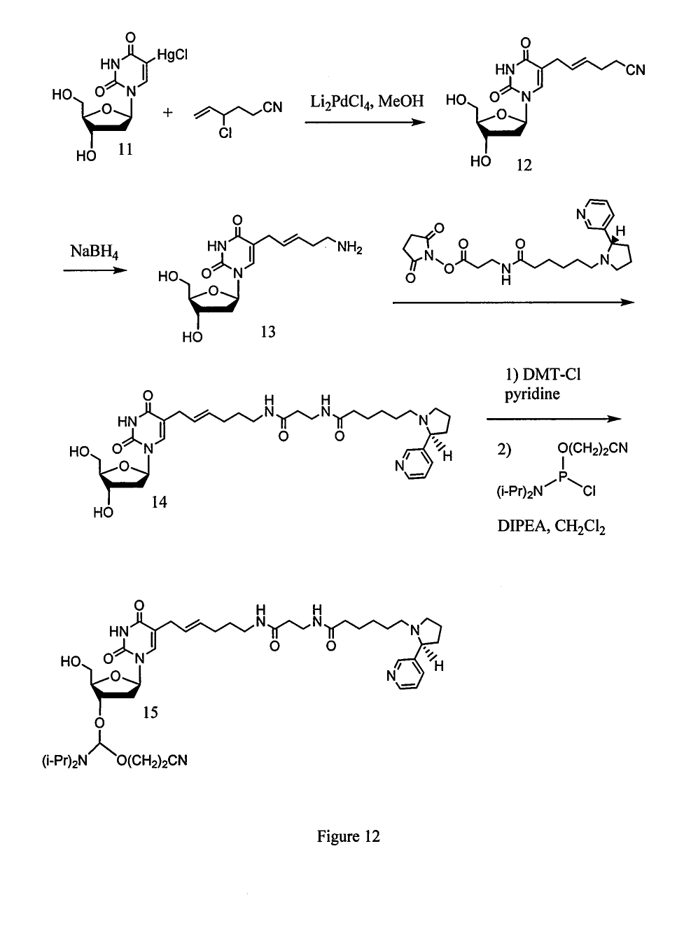

FIG. 13. Characterization of NC oligonucleotides and DNA-scaffolded NIC in their reaction to anti-NIC antibody by ELISA.

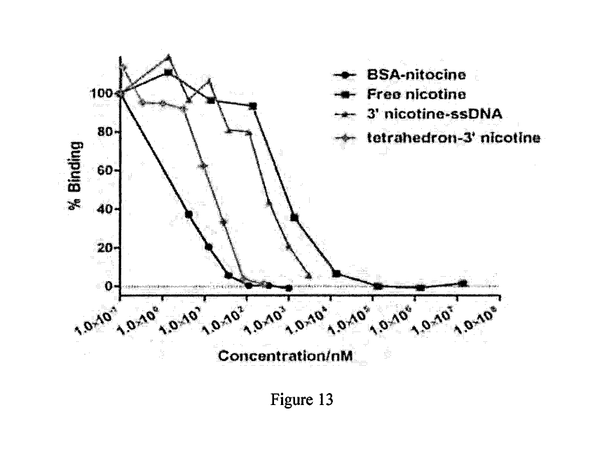

FIG. 14. Flow cytometry analyses revealing internalization of YOYO-stained DNA nanostructures.

FIG. 15. Schematic illustration of DNA-scaffolded nicotine vaccines. Helices: DNA; star: nicotine; donuts: streptavidin; and ribbons: CpG oligonucleotides. There are 16 nicotine molecules per STV protein on average, i.e. 64 nicotine molecules per vaccine complex.

FIG. 16. Time course analyses of anti-nicotine antibody responses induced by Nic-KLH+CpG (line with square); multiple immunizations with DNA-scaffolded nicotine vaccines; and priming with DNA-scaffolded nicotine vaccines (line with circle) and challenged multiple times with Nic-STV-CpG conjugates shown by line with triangle.

FIG. 17. Titer and efficacy of anti-Nic antibody responses. A. Antibody titers of individual immunized mice (n=6) after 3^{rd} or 4^{th} immunization. B. Nicotine pharmacokinetics analyses of the mice after 4^{th} immunization, where nicotine levels in blood (top) and brain were analyzed. N=6, except the last column. Data is presented as mean+SD *P<0.05, ** p<0.01: ***p<0.001 compared to controls.

FIG. 18. Structures of certain nicotine haptens

FIG. 19. Synthesis of (S)-nornicotine (6).

FIG. 20. DNA Platform for Nicotine Vaccines. A. DNA-scaffolded STV-Nicotine-CpG: STV-Nic conjugates are incorporated into a DNA-assembly with CpG. B. Nicotine-modified nucleotides for incorporation into DNA at defined positions. As used herein, donuts/globules represent STV, stars represent nicotine, helices represent DNA, and ribbons represent CpGs.

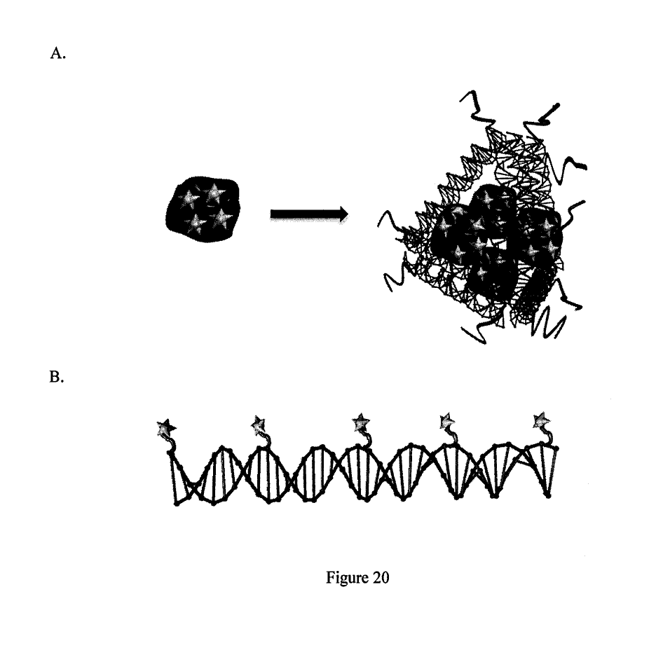

FIG. 21. STV-Nicotine Conjugates. Scheme depicting coupling reaction.

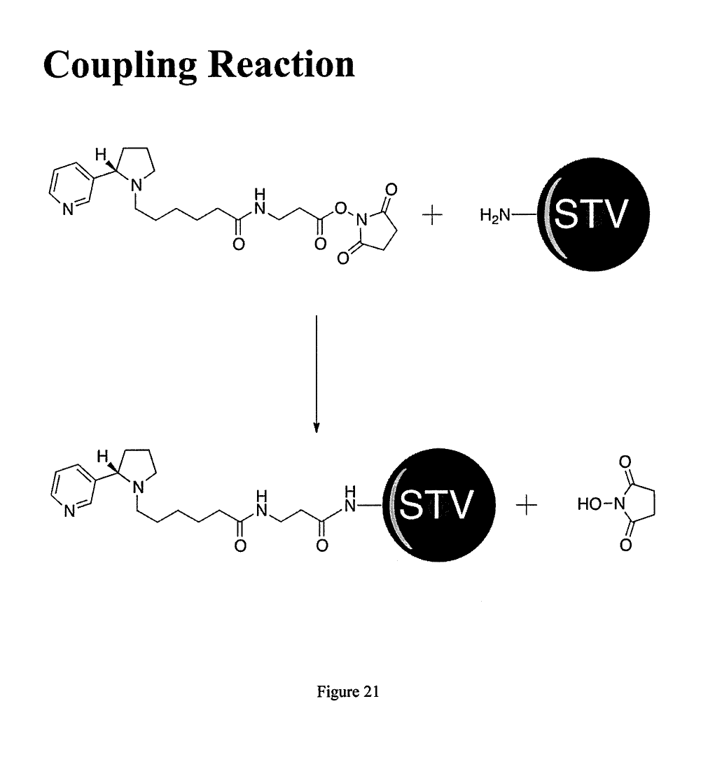

FIG. 22. Illustrations of STV-Nicotine Conjugates.

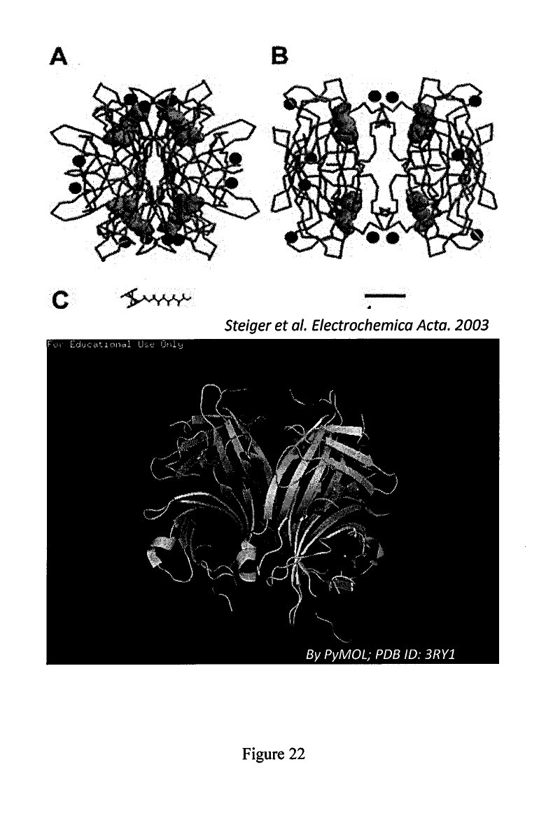

FIG. 23. Characterization of Assembled Nicotine-Vaccine.

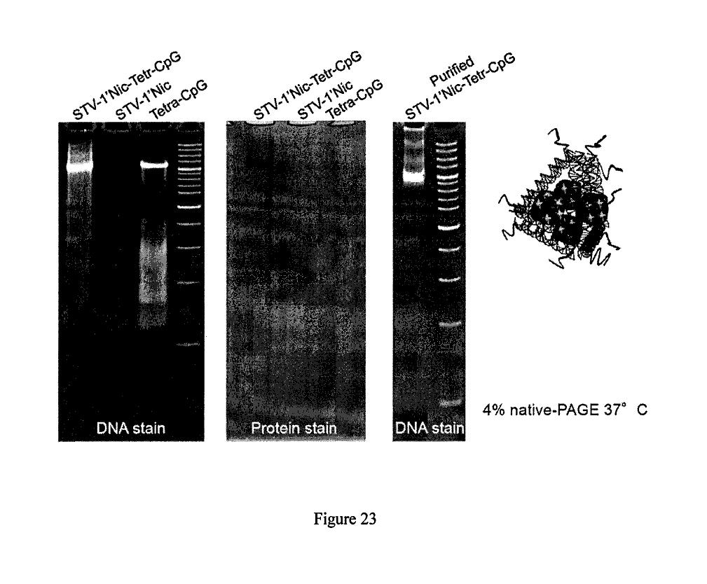

FIG. 24. Accessibility of Nicotine Displayed by DNA-particles: competition ELISA by Nic or Nic-conjugates.

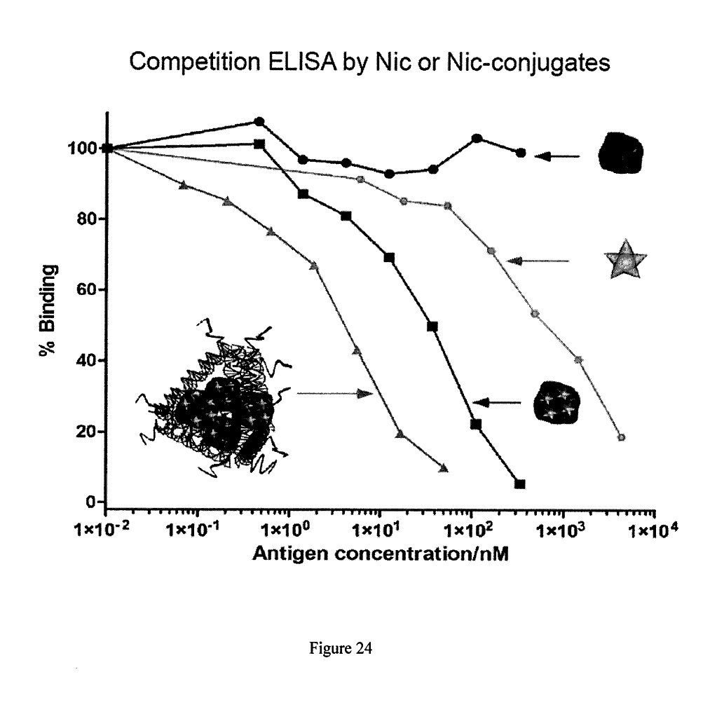

FIG. 25. Anti-nicotine antibody responses A. 8 days; and B. 25 days post 2^{nd} immunization.

FIG. 26. Anti-nicotine antibody responses 8, 25 and 39 days post 2^{nd} immunization.

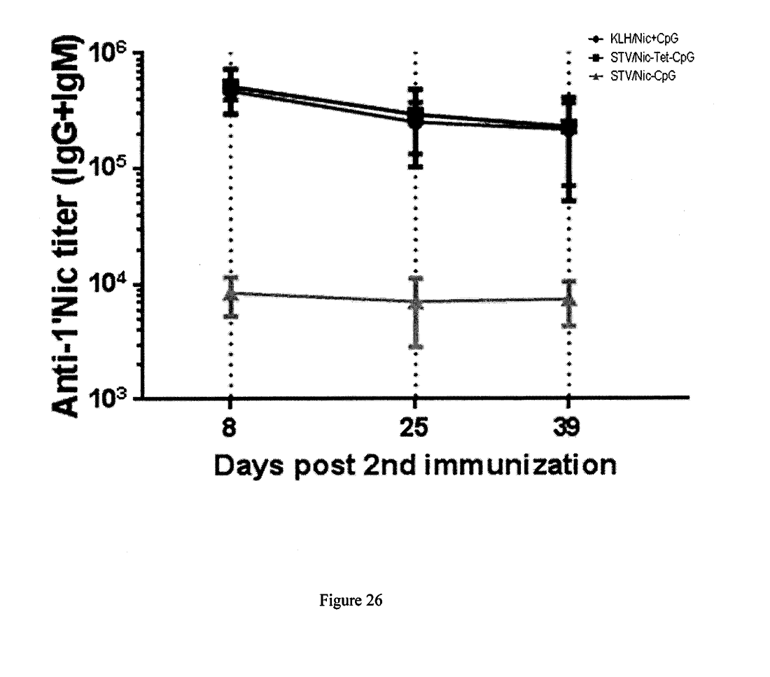

FIG. 27. Relative Ab Binding Affinity to Free Nicotine. Relative affinity was determined to be 10-15 μM. A. Competition ELISA by free nicotine. B. IC50.

FIG. 28. Illustration of a DNA-Scaffolded Nicotine Vaccine. As used herein, donuts/globules represent STV, stars represent nicotine, helices represent DNA, and small ribbons represent CpGs and large ribbons represent T-cell peptides. Nicotine may be linked to STV or may be linked the DNA through a nicotine-modified nucleotide.

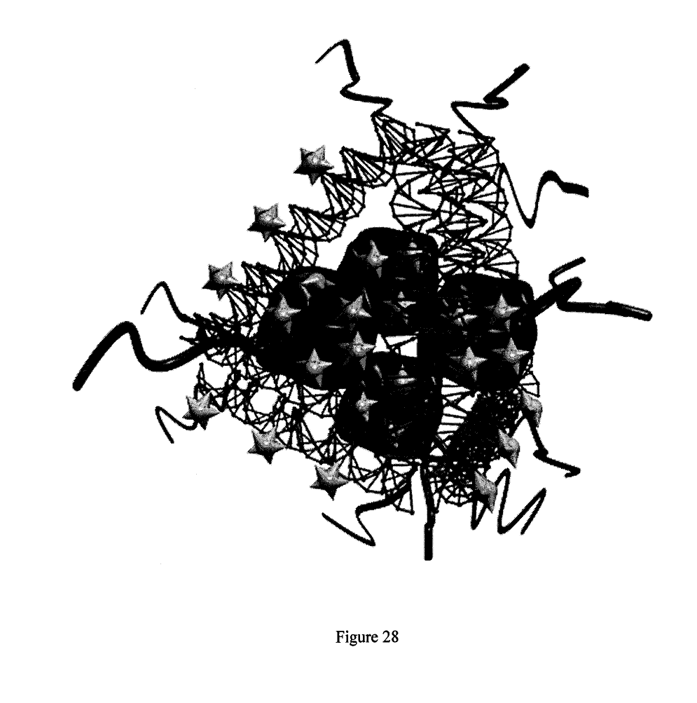

### DETAILED DESCRIPTION

DNA is a type of highly programmable molecules and proven to be an ideal building material to construct nano-devices with precise control over 3-D configurations and have demonstrated immunogenic effectiveness as a scaffold for protein antigens. The present invention applies this technology to rationally design and assemble DNA-assembled nicotine-vaccines (DNA-Nic). Data shows that after primary immunization DNA-Nic vaccine complexes induce anti-Nic antibody production in mice which are useful for treating nicotine addiction and/or prevention in mammal, in particular, humans.

### DNA-Nanostructures

The present technology utilizes DNA nanostructures as a synthetic platform for vaccine construction. Specifically, the DNA-nanostructures may be used as scaffolds to assemble nicotine as an antigenic component.

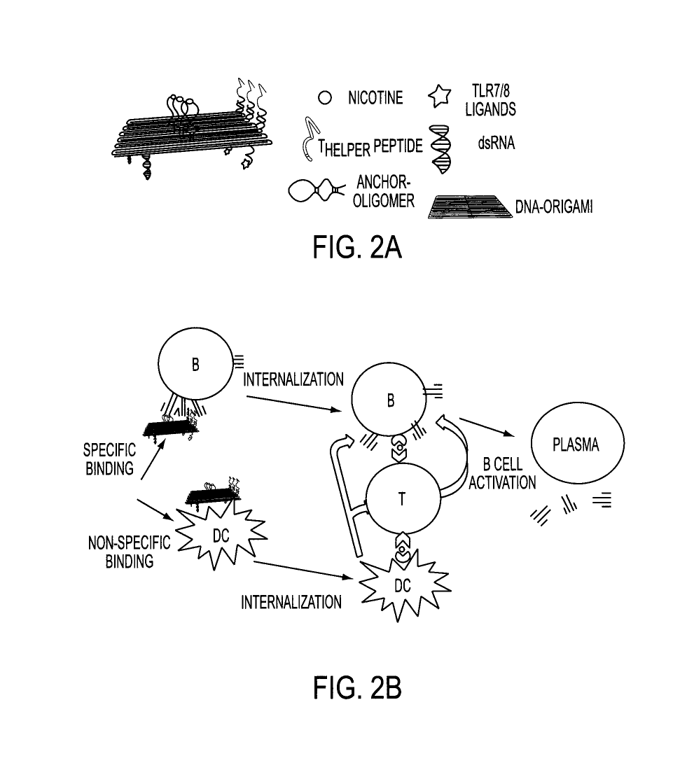

Nicotine or nicotine analogue targeting moieties may be conjugated to the DNA nanostructures at various points of attachment with various linker groups. The immunogenicity of protein-carrier/NIC conjugates has been evaluated with respect to NIC linker position (5). In addition to the pyrrolidine N linker position that was initially reported by Janda et al (4), the Pentel et al has characterized 3 NIC haptens with varying linker positions (5), two of which are shown in FIG. 2. The 3′ linker position is used in NicVax, which has shown proof of efficacy in Phase II and III clinical trials (14). The C6 and pyrrolidine N linker positions are comparably effective in rats as assessed by pharmacokinetic assays (5). In addition, the Pentel laboratory has shown that each of these linker positions stimulates distinct B cell populations, in effect acting as independent vaccines and generating non-overlapping populations of antibodies (5). As a result, it is possible to combine these vaccines without mutual interference and obtain greater serum antibody concentrations and pharmacokinetic effects than for one vaccine alone.

In certain embodiments, the nicotine targeting moiety is selected from the group consisting of:

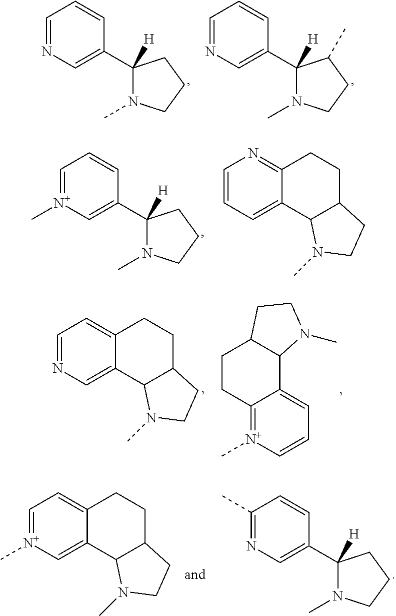

In certain embodiments, the nicotine targeting moiety is conjugated to the 5-position of a thymine nucleobase of the DNA-nanostructure. In certain embodiments, the nicotine targeting moiety is selected from the group consisting of:

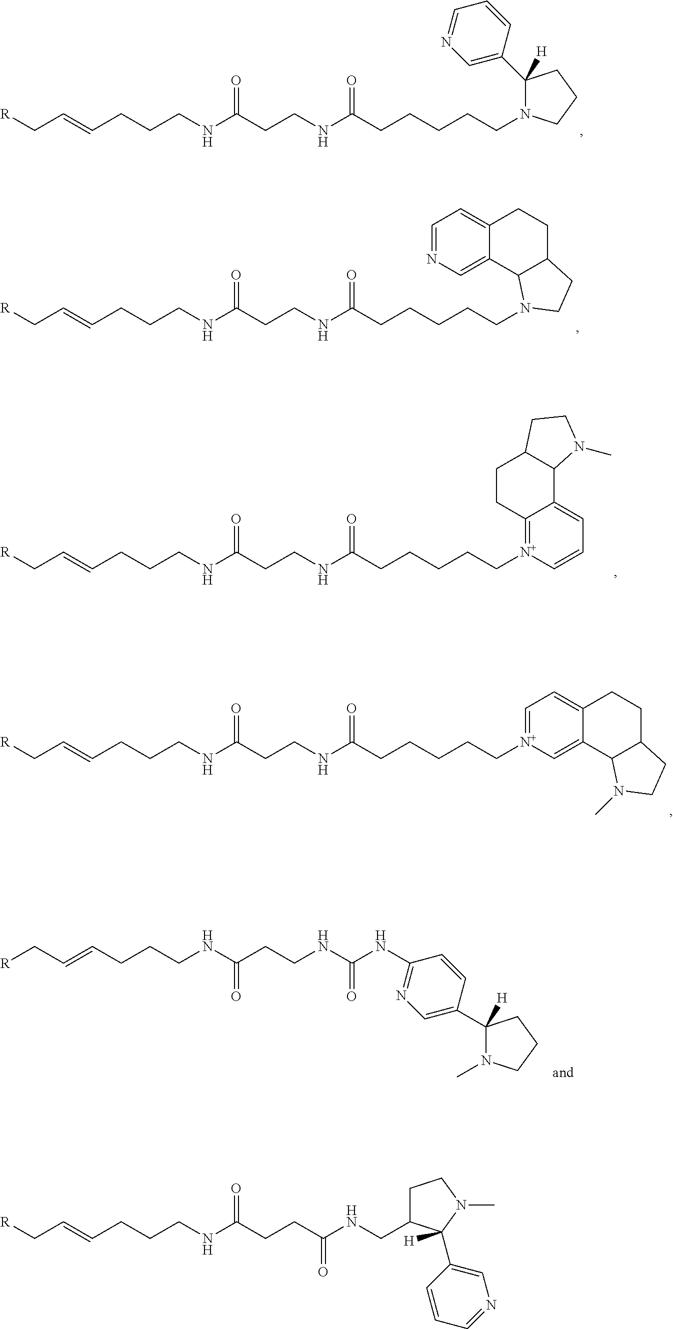

wherein R is the 5-position of a thymine nucleobase in the DNA-nanostructure.

In certain embodiments, the nicotine targeting moiety is conjugated to the 5-position of a thymine nucleobase of the DNA-nanostructure. In certain embodiments, the nicotine targeting moiety is selected from the group consisting of:

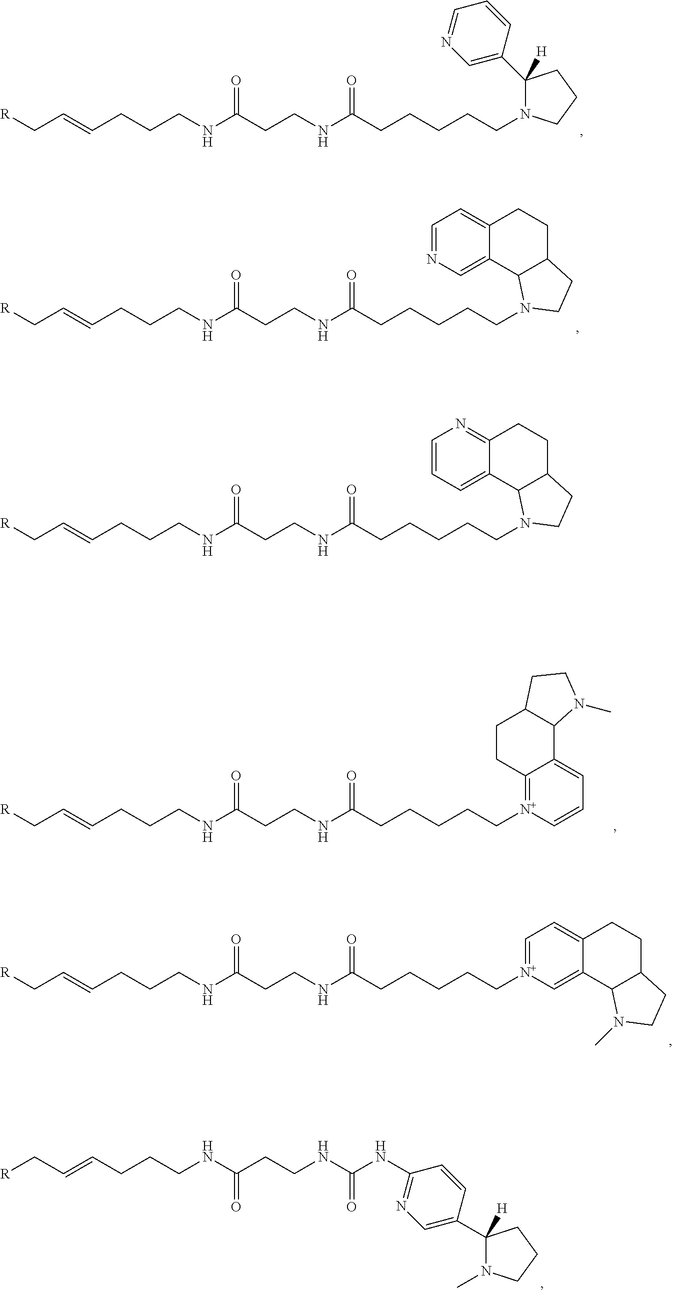

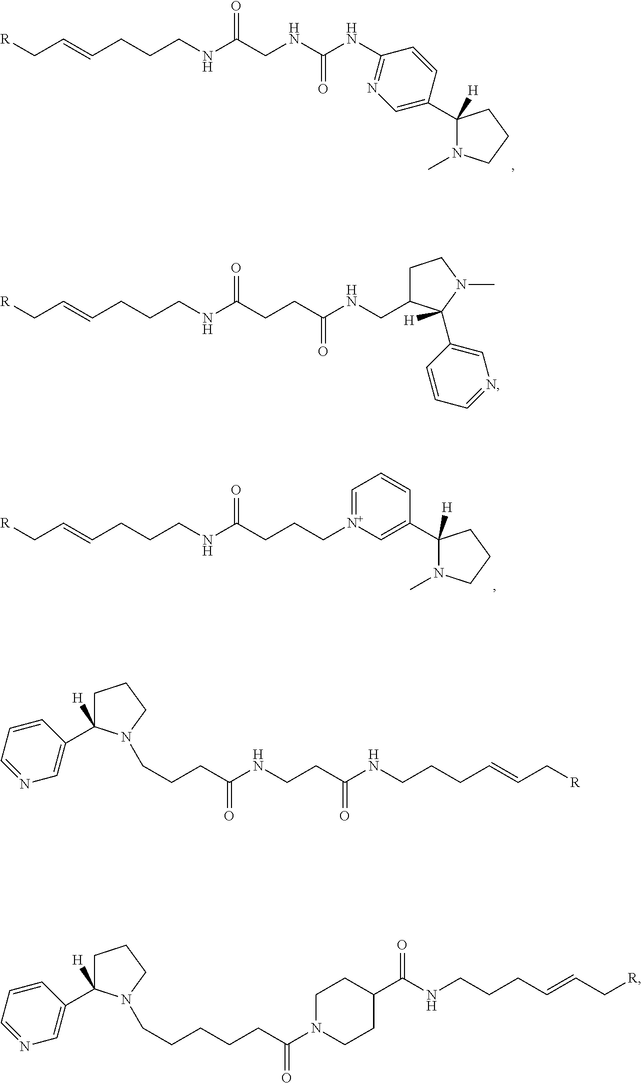

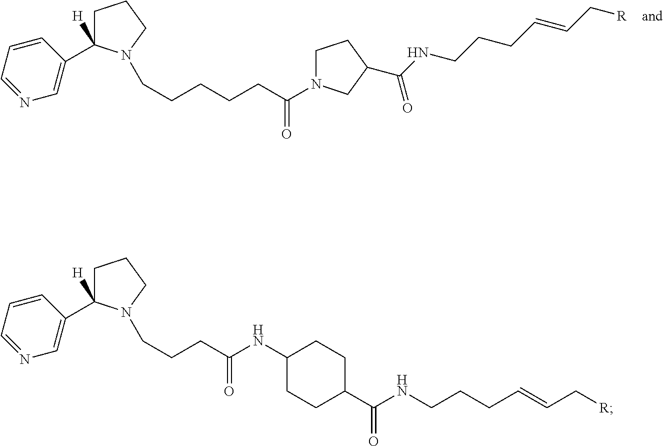

wherein R is the 5-position of a thymine nucleobase in the DNA-nanostructure.

In certain embodiments, the targeting moiety is selected from

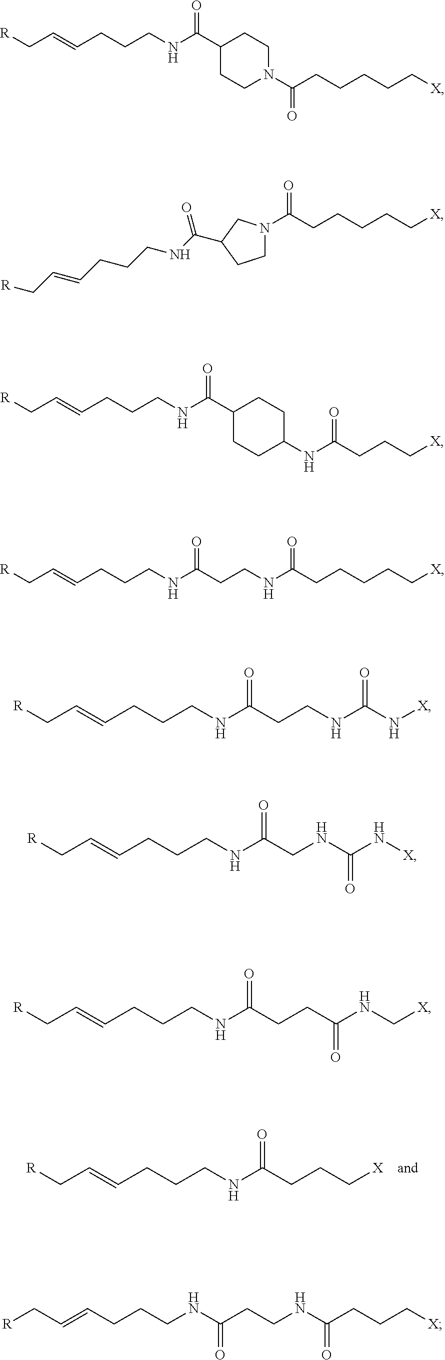

wherein X is a nicotine moiety as described herein

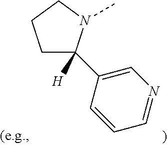

and R is the 5-position of a thymine nucleobase in the DNA-nanostructure. When attached to a carbon atom of the nicotine moiety, it is understood that in certain embodiments, the linker portion of the targeting moiety may be attached through a heteroatom, for example, through a nitrogen as shown in the following structure:

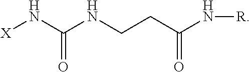

In certain embodiments, the nicotine targeting moiety is conjugated to streptavidin. Accordingly, in certain embodiments, one or more of the oligonucleotides in the DNA-nanostructure are biotinylated, which allows subsequent binding to streptavidin (i.e., the nicotine targeting moiety is linked to the DNA-nanostructure through a streptavidin-biotin linkage). In certain embodiments, one or more independently selected nicotine targeting moieties are conjugated to a streptavidin tetramer (e.g., 1, 2, 3, 4, 5, 6, 7, 8, 9, 10, 11, 12, 13, 14, 15, 16, 17, 18, 19, 20 or more). In certain embodiments, 12-16 independently selected nicotine targeting moieties are conjugated to a streptavidin tetramer. In certain embodiments, more than one streptavidin tetramer conjugated to at least one nicotine targeting moiety is linked to the DNA-nanostructure (e.g., 2, 3, 4, 5, 6, 7, 8, 9, 10, 11, 12, 13, 14, 15, 16, 17, 18, 19, 20, or more). In certain embodiments, four streptavidin tetramers are linked to the DNA-nanostructure, wherein the nicotine targeting moiety/moieties are conjugated to the streptavidin. In certain embodiments, the nicotine targeting moieties are the same. In certain embodiments, the nicotine targeting moieties are different.

In certain embodiments, the nicotine targeting moiety is selected from the group consisting of:

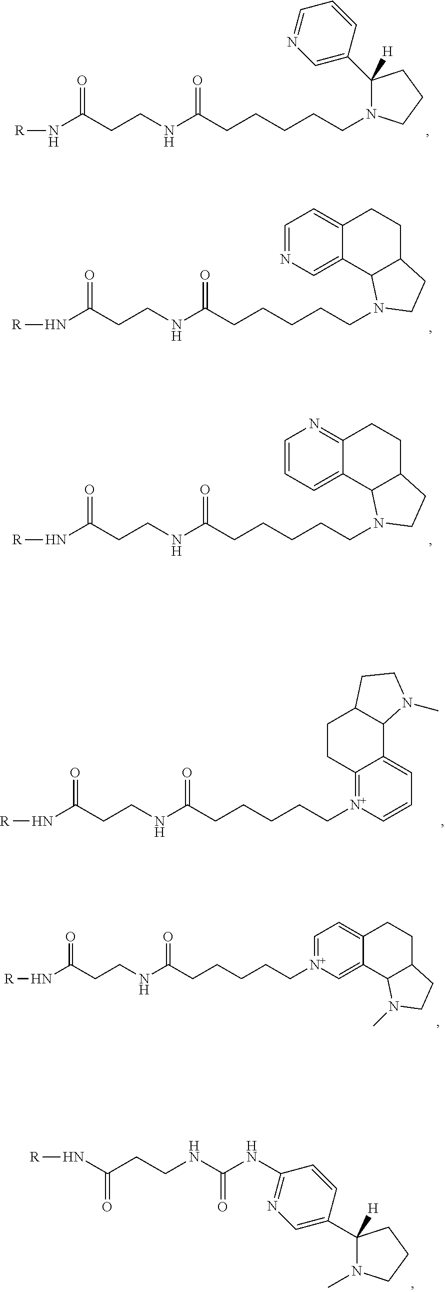

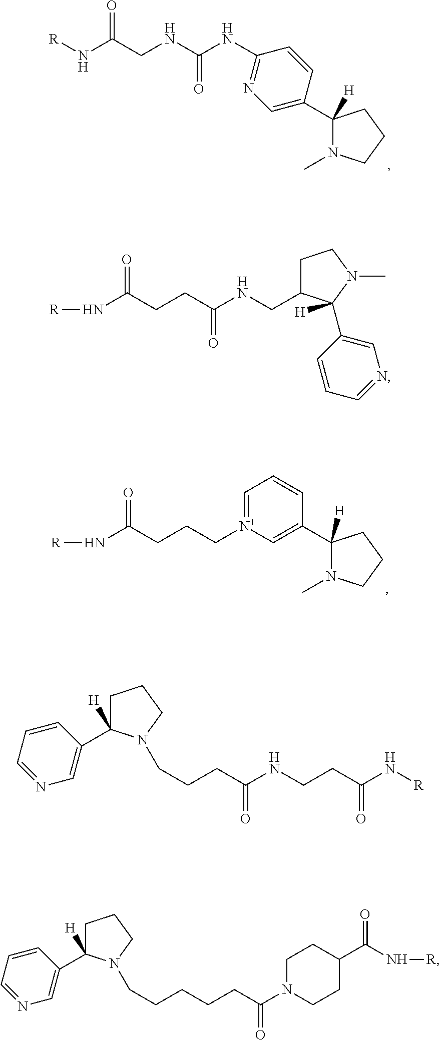

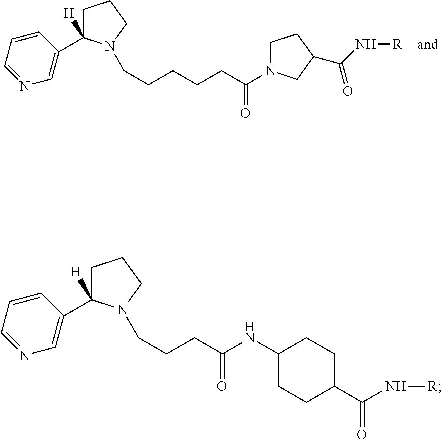

wherein R is streptavidin. It is to be understood that as shown in the above structures that the group NH—R is a residue of streptavidin.

In certain embodiments, the targeting moiety is selected from

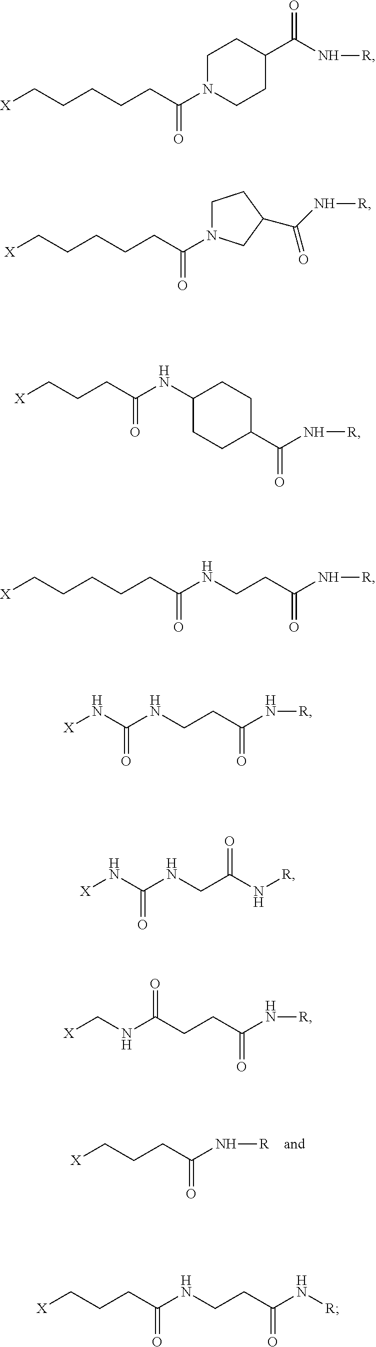

wherein X is a nicotine moiety as described herein

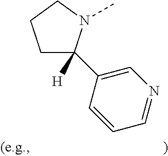

and R is streptavidin. It is to be understood that as shown in the above structures that the group NH—R is a residue of streptavidin. Additionally, when attached to a carbon atom of the nicotine moiety, it is understood that in certain embodiments, the linker portion of the targeting moiety may be attached through a heteroatom, for example, through a nitrogen as shown in the following structure:

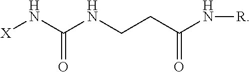

The invention also provides processes and intermediates disclosed herein that are useful for preparing compounds of the invention as described herein (i.e., a compound comprising a DNA-nanostructure and at least one targeting moiety, wherein the at least one targeting moiety is linked to the DNA-nanostructure; and wherein the at least one targeting moiety is nicotine or an analogue thereof) (see, e.g., the Examples and Figures). For example, intermediates useful for preparing a compound of the invention include:

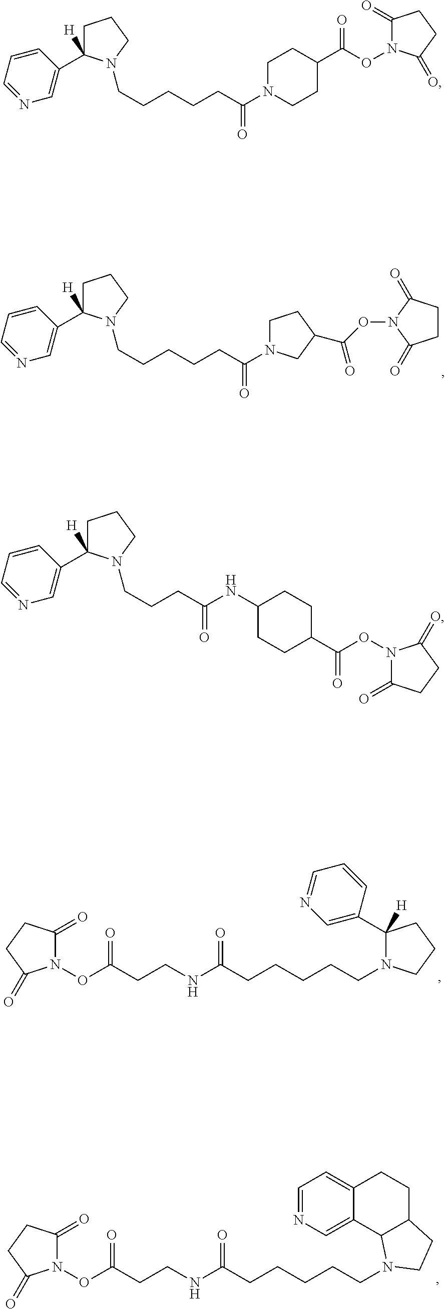 [SMILES: O=C(ON1C(=O)CCC1=O)C1CCN(C(=O)CCCCCN2CCC[C@H]2c2cccnc2)CC1 ; O=C(ON1C(=O)CCC1=O)C1CCN(C(=O)CCCCCN2CCC[C@H]2c2cccnc2)C1 ; O=C(CCCN1CCC[C@H]1c1cccnc1)NC1CCC(C(=O)ON2C(=O)CCC2=O)CC1 ; O=C(CCCCCN1CCC[C@H]1c1cccnc1)NCCC(=O)ON1C(=O)CCC1=O ; O=C(CCCCCN1CCC2CCc3ccncc3C21)NCCC(=O)ON1C(=O)CCC1=O]

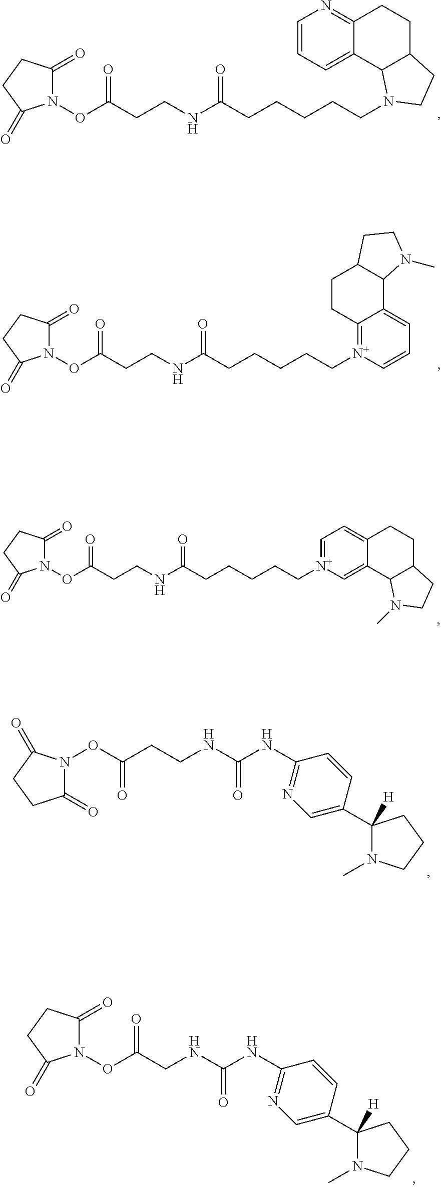 [SMILES: O=C(CCCCCN1CCC2CCc3ncccc3C21)NCCC(=O)ON1C(=O)CCC1=O ; CN1CCC2CCc3c(ccc[n+]3CCCCCC(=O)NCCC(=O)ON3C(=O)CCC3=O)C21 ; CN1CCC2CCc3cc[n+](CCCCCC(=O)NCCC(=O)ON4C(=O)CCC4=O)cc3C21 ; CN1CCC[C@H]1c1ccc(NC(=O)NCCC(=O)ON2C(=O)CCC2=O)nc1 ; CN1CCC[C@H]1c1ccc(NC(=O)NCC(=O)ON2C(=O)CCC2=O)nc1]

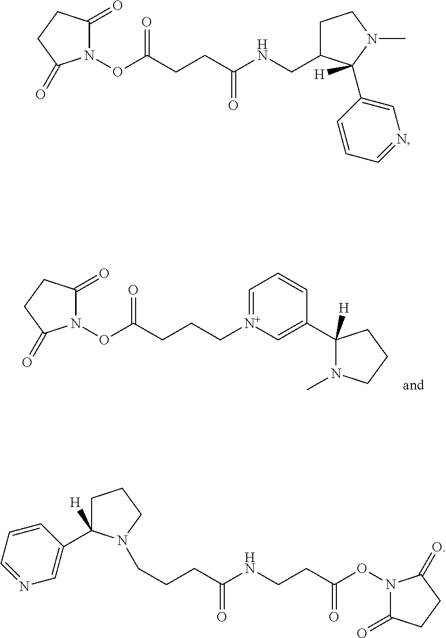 [SMILES: CN1CCC(CNC(=O)CCC(=O)ON2C(=O)CCC2=O)[C@H]1c1cccnc1 ; CN1CCC[C@H]1c1ccc[n+](CCCC(=O)ON2C(=O)CCC2=O)c1 ; O=C(CCCN1CCC[C@H]1c1cccnc1)NCCC(=O)ON1C(=O)CCC1=O]

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

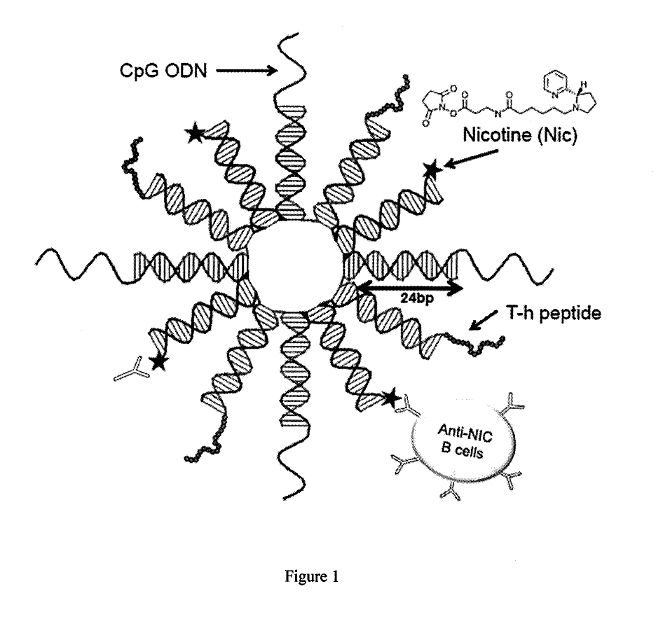

In certain embodiments, the DNA-nanostructures may be DNA-branches or DNA-tetrahedrons. In certain embodiments the DNA-nanostructures may be DNA-branches (e.g., comprising four, eight or twelve oligonucleotides). In certain embodiments, the DNA-nanostructure is a DNA-tetrahedron. These DNA-nanostructures may be prepared by methods known in the art. For example, the DNA-branches are assembled based on the concept of base-pairing; no specific sequence is required; however, the sequences of each oligonucleotide must be partially complementary to certain other oligonucleotides to enable hybridization of all strands. For example, as shown in FIG. 1, four oligonucleotides with partial complementary sequences may be used to construct the DNA-branch. In certain embodiments one of the oligonucleotides is a CpG oligonucleotide. CpG oligodeoxynucleotides (CpG ODN) are short single stranded (ss) DNA that contain “C-P(phosphodiester or phosphorothioate)-G” structure. In certain embodiments, one or more of the oligonucleotides in the DNA-nanostructure is biotinylated, which allows subsequent binding to an antigen, such as streptavidin. The DNA strands may be assembled by heating at 95° C. and then annealing at room temperature. In certain embodiments, the DNA-tetrahedrons may be prepared by methods described in Zhang, et al., Chem Commun, 46, 6792-6794 (2010) and He et al., Nature, 2008, 452, 198, which are herein incorporated by reference.

The length of each oligonucleotide or DNA strand is variable and depends on, for example, the type of nanostructure and the number of targeting moieties to be linked. In certain embodiments, the oligonucleotide or DNA strand is about 15 nucleotides in length to about 3000 nucleotides in length, such as 15 to 100 nucleotides, or 600-800 nucleotides.

For use in the present invention, the nucleic acids can be synthesized de novo using any of a number of procedures well known in the art. For example, the cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054,1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated oligonucleotide synthesizers available in the market.

DNA nanostructures used in the invention may be 8-arm and 12-arm branched DNA “ball” (32), in addition to the tetrahedron DNA. Each of these structures offers the ability to display pre-determined numbers of antigenic determinants, i.e., the ratio of NIC:Th-epitope:adjuvant. In certain embodiment the ratio of NIC:Th-epitope:adjuvant is 3:3:2, 4:4:4 and 12:12:12 for the 8-arm, 12-arm and tetrahedron DNA structures, respectively. The precision with which we can control the conjugation of NIC to defined positions within a known oligonucleotide underscores the ability to present NIC haptens on the surface of the DNA nanostructures. In certain embodiments, the DNA nanostructures of the invention incorporate CpG-ODN and TLR7/8-ligands as adjuvants, both of which are known to activate murine and human B cells (27,33,34). TLR9-ligand has been reported for clinical use in vaccines and immunotherapeutics and TLR7/8-ligands such as Imiquimod and resiquimod (R848) which have been approved by the FDA for their stand-alone use (35), and were reported to increase the immunogenicity of another synthetic NIC vaccine (28). In certain embodiments, the Th-epitope is a pan HLADR epitope peptide (PADRE). PADRE is a well-known promiscuous peptide that can bind to a wide spectrum of MHC-II, including some human and rodent cells. It has been shown to help activate B cell responses against haptens or nonimmunogenic antigens (25,45). In certain embodiments, The Th-epitope is a peptide that is absent from the human proteome. In certain embodiments the Th-epitope is a peptide of about 5 to 10 residues, and in a particular embodiment is 5 residues.

Alternatively, DNA nanostructures of the invention may be DNA-origami structures that permit a high level of epitope insertion (44), e.g., 50-100 copies. In certain embodiments, the DNA nanostructure is a DNA-origami structure having NIC:Th-epitope:adjuvant ratio of 30:30:30. In certain embodiments, the DNA nanostructure is a DNA-origami structure having NIC:Th-epitope:adjuvant ratio of 50:20:20.

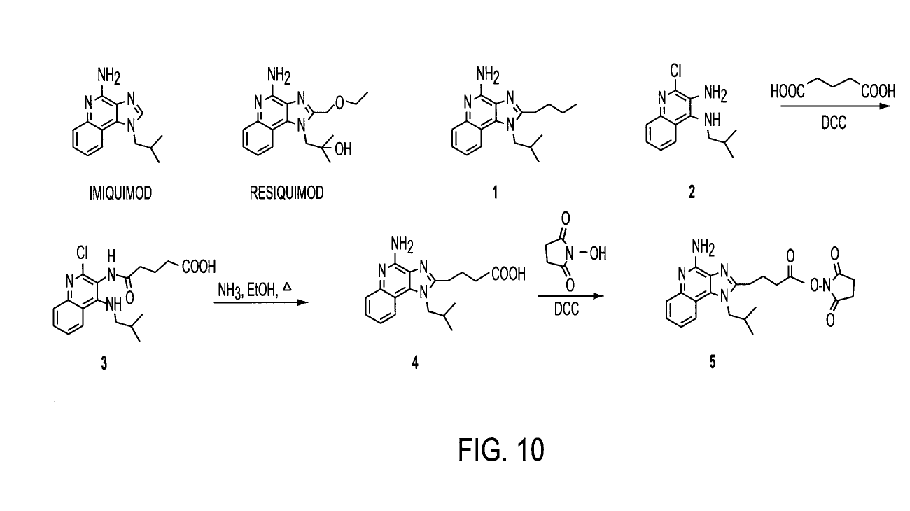

TLR7/8 ligands, imiquimod and its structurally related compound (R848), are shown in FIG. 10. These two TLR7/8 ligands have been reported to function as effective adjuvants in humoral immune responses (37). In order to attach a compound of this type to DNA, it is necessary to identify and prepare an active analogue having an appropriate functional group. Compound 1 in FIG. 10 is a hybrid of imiquimod and resiquimod that has been prepared and shown to increase adjuvant potency 50 times more than imiquimod. Substitution of the imidazole ring with a variety of other aliphatic and aromatic substituents generally retained IFN-inducing activity (38). Thus compound 4 can be used as an analogue of 1 to prepare DNA nanostructures of the invention that retain the TLR7/8 targeting moiety activity. Specifically, compound 4 will be prepared starting from known intermediate 2 using a route that has afforded many similar derivatives (38). DCC mediated coupling with glutaric acid will afford amide 3. Subsequent treatment with methanolic ammonia will effect replacement of the chloro substituent and imidazole ring formation, affording the desired compound 4. Activation as the N-hydroxysuccinimide ester will then lead to 5, affording a deoxynucleoside phosphoramidate suitable for preparing derivatized DNA.

In certain embodiments, at least one targeting moiety may be linked to the DNA-nanostructures. In certain embodiments, the composition comprises at least two targeting moieties (e.g. 2, 3, 4, 5, 6, 7, 8, 9, 10, etc.). In certain embodiments the targeting moieties are the same and in certain embodiments, the targeting moieties are different.

These targeting moieties may be assembled onto DNA-nanostructures at designated positions, i.e., in desired multi-valence, appropriate stoichiometry, and spatial orientations to elicit strong memory B cell responses. In certain embodiments, the targeting moieties are linked to the DNA nanostructure in polymeric forms. In certain embodiments, the polymeric form is trimeric.

In certain embodiments, the compound comprises a DNA-nanostructure and at least one nicotine targeting moiety as described herein, wherein the at least one targeting moiety is linked to the DNA-nanostructure, and wherein the DNA-nanostructure is a DNA-tetrahedron. In certain embodiments, the at least one nicotine targeting moiety is conjuaged to streptavidin (e.g., a streptavidin tetramer). In certain embodiments, one or more independently selected nicotine targeting moieties are conjugated to a streptavidin tetramer (e.g., 1, 2, 3, 4, 5, 6, 7, 8, 9, 10, 11, 12, 13, 14, 15, 16, 17, 18, 19, 20 or more). In certain embodiments, the at least one nicotine targeting moiety is linked to the DNA-nanostructure through a streptavidin-biotin linkage. In certain embodiments, four streptavidin tetramers are linked to the DNA-nanostructure, wherein the nicotine targeting moiety/moieties are conjugated to the streptavidin (e.g., 12-16 nicotine targeting moieties per streptavidin tetramer). In certain embodiments, the compound further comprises a CpG oligonucleotide. For example, in certain embodiments, one of the oligonucleotides of the DNA-nanostructure may be a CpG oligonucleotide, or in other embodiments, the CpG oligonucleotide may be linked to the DNA-nanostructure (e.g., through complementary base pairing). In certain embodiments, the CpG oligonucleotide is linked to streptavidin (e.g., through a streptavidin-biotin linkage). In certain embodiments, the CpG oligonucleotide is not linked to the DNA-nanostructure.

In certain embodiments the targeting moiety is selected from the group consisting of antigens, aptamers, shRNAs and combinations thereof.

### Antigens

In certain embodiments the DNA-nanostructure includes, in addition to nicotine or a nicotine analogue, at least one targeting moiety that is an antigen. As one skilled in the art will appreciate, it is not necessary to use the entire antigen. A selected portion of the antigen, for example the epitope, can be used.

As one skilled in the art will also appreciate, it is not necessary to use an antigen that is identical to a native antigen. The modified antigen can correspond essentially to the corresponding native antigen. As used herein “correspond essentially to” refers to an epitope that will elicit an immunological response at least substantially equivalent to the response generated by a native antigen. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest.

In certain embodiments the antigen is selected from the group consisting of a B-cell epitope, a T-cell epitope, a Tₕₑₗₚₑᵣ epitope, a peptide, or a T-helper peptide

### Aptamers

In certain embodiments, the at least one targeting moiety, in addition to nicotine or an analogue thereof, is an aptamer.

Aptamers are single stranded oligonucleotides that can naturally fold into different 3-dimensional structures, which have the capability of binding specifically to biosurfaces, a target compound or a moiety. The term “conformational change” refers to the process by which a nucleic acid, such as an aptamer, adopts a different secondary or tertiary structure. The term “fold” may be substituted for conformational change.

Aptamers can have low immunogenicity. They can easily be synthesized in large quantities at a relatively low cost and are amendable to a variety of chemical modifications that confer both resistance to degradation and improved pharmacokinetics in vivo. The smaller size of aptamers compared with that of antibodies (<15 kDa versus 150 kDa) facilitates their in vivo delivery by promoting better tissue penetration.

Aptamers have advantages over more traditional affinity molecules such as antibodies in that they are very stable, can be easily synthesized, and can be chemically manipulated with relative ease. Aptamer synthesis is potentially far cheaper and reproducible than antibody-based diagnostic tests. Aptamers are produced by solid phase chemical synthesis, an accurate and reproducible process with consistency among production batches. An aptamer can be produced in large quantities by polymerase chain reaction (PCR) and once the sequence is known, can be assembled from individual naturally occurring nucleotides and/or synthetic nucleotides. Aptamers are stable to long-term storage at room temperature, and, if denatured, aptamers can easily be renatured, a feature not shared by antibodies. Furthermore, aptamers have the potential to measure concentrations of ligand in orders of magnitude lower (parts per trillion or even quadrillion) than those antibody-based diagnostic tests. These characteristics of aptamers make them attractive for diagnostic applications.

Aptamers are typically oligonucleotides that may be single stranded oligodeoxynucleotides, oligoribonucleotides, or modified oligodeoxynucleotide or oligoribonucleotides. The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues α-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4,N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpseudouracil; 1-methylguanine; 1-methylcytosine.

Aptamers may be synthesized using conventional phosphodiester linked nucleotides and synthesized using standard solid or solution phase synthesis techniques, which are known in the art. Linkages between nucleotides may use alternative linking molecules. For example, linking groups of the formula P(O)S, (thioate); P(S)S, (dithioate); P(O)NR′2; P(O)R; P(O)OR6; CO; or CONR′2 wherein R is H (or a salt) or alkyl (1-12C) and R6 is alkyl (1-9C) is joined to adjacent nucleotides through —O— or —S—.

In certain embodiments, modifications are made to the aptamer(s). Additional modifications to the aptamer include 2′O-methyl modification of the pyrimidines. In other embodiments, all of the nucleotides in the aptamer are 2′O-methyl modified. Alternatively, the pyrimidines, or all the nucleotides, may be modified with 2′fluoro substituents (both pyrimidines and purines). Additional modifications to the nucleotides in the aptamer include large molecular weight conjugates like pegylation, lipid-based modifications (e.g., cholesterol) or nanoparticles (e.g., PEI or chitosan) to improve the pharmacokinetic/dynamic profile of the chimera.

In certain embodiments, modifications are introduced into the stem sequence in the aptamer. Different nucleotides can be used as long as the structure of the stem is retained.

In certain embodiments, the aptamer molecule is about 10 nucleotides in length to about 1,000 nucleotides in length. In certain embodiments, the aptamer molecule is not more than 500 nucleotides in length. In certain embodiments, the aptamer molecule is not more than 100 nucleotides in length. In certain embodiments, the total scaffold of the aptamer is about 80 nucleotides. In certain embodiments, the binding region is about 20-60 nucleotides, such as about 40 nucleotides.

### RNAi Molecules

In certain embodiments, the at least one targeting moiety, in addition to nicotine or analogue thereof, is an RNA interference (RNAi) molecule. In certain embodiments, the RNAi molecule is shRNA, siRNA or miRNA.

A small hairpin RNA or short hairpin RNA (shRNA) is a sequence of RNA that makes a tight hairpin turn that can be used to silence gene expression via RNA interference.

### Detection Means

In certain embodiments, the composition further comprises a detection means. In certain embodiments, the detection means is linked to the DNA nanostructure.

In certain embodiments, the targeting moiety may comprise a detection means. In certain embodiments, the targeting moiety is operably linked to the detection means.

A number of “molecular beacons” (such as fluorescence compounds) can be attached to the DNA nanostructure or targeting moiety to provide a means for signaling the presence of and quantifying a target chemical, cell or biological agent, for example, R-Phycoerythrin (PE). Other exemplary detection labels that could be attached to the targeting moiety include biotin, any fluorescent dye, amine modification, horseradish peroxidase, alkaline phosphatase, etc. In certain embodiments, the detection means is linked to the DNA nanostructure, and in certain embodiments, the detection means is linked to the targeting moiety.

### CpG Oligonucleotides and Other Adjuvants

In certain embodiments, compositions of the invention further comprise at least one adjuvant. In certain embodiments, the adjuvant is a targeting moiety linked to the DNA nanostructure. In certain embodiments, compositions of the invention further comprise at least two adjuvants (e.g. 2, 3, 4, 5, 6, 7, 8, 9, 10, etc.). In certain embodiments, the adjuvants are the same and in certain embodiments, the adjuvants are different. In certain embodiments, all of the adjuvants are linked to the DNA-nanostructure. In certain embodiments, none of the adjuvants are linked to the DNA-nanostructure. In certain embodiments, one or more of the adjuvants are linked to the DNA-nanostructure and one or more of the adjuvants are not linked to the DNA-nanostructure. When an adjuvant(s) is not linked to the DNA-nanostructure, the composition can be administered before, after, and/or simultaneously with the adjuvant(s).

A conventional “adjuvant” is any molecule or compound that nonspecifically stimulates the humoral and/or cellular immune response. They are considered to be nonspecific because they only produce an immune response in the presence of an antigen. Adjuvants allow much smaller doses of antigen to be used and are essential to inducing a strong antibody response to soluble antigens.

Immunostimulatory oligonucleotides, which directly activate lymphocytes and co-stimulate an antigen-specific response, are fundamentally different from conventional adjuvants (e.g., aluminum precipitates), which are inert when injected alone and are thought to work through absorbing the antigen and thereby presenting it more effectively to immune cells.

In certain embodiments, an adjuvant may be an oligonucleotide containing at least one immunostimulatory CpG motif. Additional suitable adjuvants include but are not limited to surfactants, e.g., hexadecylamine, octadecylamine, lysolecithin, dimethyldioctadecylammonium bromide, N,N-dioctadecyl-N′—N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, aimethylglycine, tuftsin, oil emulsions, aluminum (alum), aluminum hydroxide, incomplete Freud's adjuvant, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). CpG

### Oligonucleotides

An oligonucleotide containing at least one immunostimulatory CpG motif can be used to activate the immune response. CpG DNA for use as a vaccine adjuvant is known in the art and described, for example, in Bode et al., Expert. Rev. Vaccines, 10(4), 499-511 (2011) and U.S. Publication 2008-0124366, which are incorporated herein by reference.

As used herein the article “a” or “an” is used to mean “one or more.” For example “an oligonucleotide” would mean “one or more oligonucleotide.”

The term “nucleic acid” or “oligonucleotide” refers to a polymeric form of nucleotides at least five bases in length. The term “oligonucleotide” includes both single and double-stranded forms of nucleic acid. The nucleotides of the invention can be deoxyribonucleotides, ribonucleotides, or modified forms of either nucleotide. Generally, double-stranded molecules are more stable in vivo, although single-stranded molecules can have increased stability when they contain a synthetic backbone.

An “oligodeoxyribonucleotide” (ODN) as used herein is a deoxyribonucleic acid sequence from about 3-1000 (or any integer in between) bases in length. In certain embodiments, the ODN is about 3 to about 50 bases in length. Lymphocyte ODN uptake is regulated by cell activation. For example, B-cells that take up CpG ODNs proliferate and secrete increased amounts of immunoglobulin. Certain oligonucleotides containing at least one unmethylated cytosine-guanine (CpG) dinucleotide activate the immune response.

A “CpG” or “CpG motif” refers to a nucleic acid having a cytosine followed by a guanine linked by a phosphate bond. The term “methylated CpG” refers to the methylation of the cytosine on the pyrimidine ring, usually occurring at the 5-position of the pyrimidine ring. The term “unmethylated CpG” refers to the absence of methylation of the cytosine on the pyrimidine ring. Methylation, partial removal, or removal of an unmethylated CpG motif in an oligonucleotide of the invention is believed to reduce its effect. Methylation or removal of all unmethylated CpG motifs in an oligonucleotide substantially reduces its effect. The effect of methylation or removal of a CpG motif is “substantial” if the effect is similar to that of an oligonucleotide that does not contain a CpG motif.

In certain embodiments the CpG oligonucleotide is in the range of about 8 to 30 bases in size, or about 15 to 20 bases in size. For use in the present invention, the nucleic acids can be synthesized de novo using any of a number of procedures well known in the art. For example, the cyanoethyl phosphoramidite method (Beaucage, S. L., and Caruthers, M. H., Tet. Lett. 22:1859, 1981); nucleoside H-phosphonate method (Garegg et al., Tet. Lett. 27:4051-4054,1986; Froehler et al., Nucl. Acid. Res. 14:5399-5407, 1986; Garegg et al., Tet. Lett. 27:4055-4058, 1986, Gaffney et al., Tet. Lett. 29:2619-2622, 1988). These chemistries can be performed by a variety of automated oligonucleotide synthesizers available in the market.

As used herein the term “palindromic sequence” means an inverted repeat (i.e., a sequence such as ABCDEE′D′C′B′A′ in which A and A′ are bases capable of forming the usual Watson-Crick base pairs. In vivo, such sequences may form double-stranded structures.

A “stabilized nucleic acid molecule” shall mean a nucleic acid molecule that is relatively resistant to in vivo degradation (e.g., via an exo- or endo-nuclease). Stabilization can be a function of length or secondary structure. Unmethylated CpG containing nucleic acid molecules that are tens to hundreds of kilobases long are relatively resistant to in vivo degradation. For shorter immunostimulatory nucleic acid molecules, secondary structure can stabilize and increase their effect. For example, if the 3′ end of a nucleic acid molecule has self-complementarity to an upstream region, so that it can fold back and form a sort of stem loop structure, then the nucleic acid molecule becomes stabilized and therefore exhibits more activity.

In certain embodiments, stabilized nucleic acid molecules of the instant invention have a modified backbone. It has been shown that modification of the oligonucleotide backbone provides enhanced activity of the CpG molecules of the invention when administered in vivo. CpG constructs, including at least two phosphorothioate linkages at the 5′ end of the oligodeoxyribonucleotide and multiple phosphorothioate linkages at the 3′ end, provided maximal activity and protected the oligodeoxyribonucleotide from degradation by intracellular exo- and endo-nucleases. Other modified oligodeoxyribonucleotides include phosphodiester modified oligodeoxyribonucleotide, combinations of phosphodiester, phosphorodithioate, and phosphorothioate oligodeoxyribonucleotide, methyiphosphonate, methylphosphorothioate, phosphorodithioate, or methylphosphorothioate and combinations thereof. The phosphate backbone modification can occur at the 5′ end of the nucleic acid, for example at the first two nucleotides of the 5′ end of the nucleic acid. The phosphate backbone modification may occur at the 3′ end of the nucleic acid, for example at the last five nucleotides of the 3′ end of the nucleic acid. Nontraditional bases such as hypoxanthine and queosine, as well as acetyl-, thio- and similarly modified forms of adenine, cytosine, guanine, thymine, and uracil can also be included, which are not as easily recognized by endogenous endonucleases. Other stabilized nucleic acid molecules include: nonionic DNA analogs, such as alkyl- and aryl-phosphonates (in which the charged oxygen moiety is alkylated). Nucleic acid molecules that contain a diol, such as tetraethyleneglycol or hexaethyleneglycol, at either or both termini are also included.

DNA containing unmethylated CpG dinucleotide motifs in the context of certain flanking sequences has been found to be a potent stimulator of several types of immune cells in vitro. (Ballas, et al., J. Immunol. 157:1840 (1996); Cowdrey, et al., J. Immunol. 156:4570 (1996); Krieg, et al., Nature 374:546 (1995)) Depending on the flanking sequences, certain CpG motifs may be more immunostimulatory for B cell or T cell responses, and preferentially stimulate certain species. When a humoral response is desired, preferred immunostimulatory oligonucleotides comprising an unmethylated CpG motif will be those that preferentially stimulate a B cell response. When cell-mediated immunity is desired, preferred immunostimulatory oligonucleotides comprising at least one unmethylated CpG dinucleotide will be those that stimulate secretion of cytokines known to facilitate a CD8+T cell response.

The immunostimulatory oligonucleotides of the invention may be chemically modified in a number of ways in order to stabilize the oligonucleotide against endogenous endonucleases. As used herein, these contain “synthetic phosphodiester backbones.” For example, the oligonucleotides may contain other than phosphodiester linkages in which the nucleotides at the 5′ end and/or 3′ end of the oligonucleotide have been replaced with any number of non-traditional bases or chemical groups, such as phosphorothioate-modified nucleotides. The immunostimulatory oligonucleotide comprising at least one unmethylated CpG dinucleotide may preferably be modified with at least one such phosphorothioate-modified nucleotide. Oligonucleotides with phosphorothioate-modified linkages may be prepared using methods well known in the field such as phosphoramidite (Agrawal, et al., Proc. Natl. Acad. Sci. 85:7079 (1988)) or H-phosphonate (Froehler, et al., Tetrahedron Lett. 27:5575 (1986)). Examples of other modifying chemical groups include alkylphosphonates, phosphorodithioates, alkylphosphorothioates, phosphoramidates, 2-O-methyls, carbamates, acetamidates, carboxymethyl esters, carbonates, and phosphate triesters. Oligonucleotides with these linkages can be prepared according to known methods (Goodchild, Chem. Rev. 90:543 (1990); Uhlmann, et al., Chem. Rev. 90:534 (1990); and Agrawal, et al., Trends Biotechnol. 10:152 (1992)). A “partially synthetic backbone” is a backbone where some of the oligonucleotides are modified, and a “completely synthetic backbone” is one where all of the oligonucleotides are modified. A “natural phosphodiester backbone” is one where the oligonucleotides have not been modified.

Other stabilized nucleic acid molecules include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acid molecules which contain diol, such as tetraethyleneglycol or hexaethyleneglycol, at either or both termini have also been shown to be substantially resistant to nuclease degradation.

A “subject” shall mean a human or vertebrate animal including a dog, cat, horse, cow, pig, sheep, goat, chicken, monkey, rat, and mouse. Nucleic acids containing an unmethylated CpG can be effective in any mammal, such as a human. Different nucleic acids containing an unmethylated CpG can cause optimal immune stimulation depending on the mammalian species. Thus an oligonucleotide causing optimal stimulation in humans may not cause optimal stimulation in a mouse. One of skill in the art can identify the optimal oligonucleotides useful for a particular mammalian species of interest.

The stimulation index of a particular immunostimulatory CpG ODN to effect an immune response can be tested in various immune cell assays. The stimulation index of the immune response can be assayed by measuring various immune parameters, e.g., measuring the antibody-forming capacity, number of lymphocyte subpopulations, mixed leukocyte response assay, lymphocyte proliferation assay. The stimulation of the immune response can also be measured in an assay to determine resistance to infection or tumor growth. Methods for measuring a stimulation index are well known to one of skill in the art. For example, one assay is the incorporation of ³H thymidine in a murine B cell culture, which has been contacted with a 20 pM of oligonucleotide for 20 h at 37° C. and has been pulsed with 1 pCi of ³H uridine; and harvested and counted 4 h later. The induction of secretion of a particular cytokine can also be used to assess the stimulation index. In one method, the stimulation index of the CpG ODN with regard to B-cell proliferation is at least about 5, at least about 10, at least about 15, or even at least about 20 (as described in detail in U.S. Pat. No. 6,239,116), while recognizing that there are differences in the stimulation index among individuals.

The term “polynucleotide” or “nucleic acid sequence” refers to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The nucleotides of the invention can be ribonucleotides, deoxyribonucleotides, or modified forms of either nucleotide. The term includes single and double stranded forms of DNA.

Methods for Making Immunostimulatory Nucleic Acids

For use in the instant invention, nucleic acids can be synthesized de novo using any of a number of procedures well known in the art. For example, the β-cyanoethyl phosphoramidite method (S. L. Beaucage and M. H. Caruthers, 1981, Tet. Let. 22:1859); nucleoside H-phosphonate method (Garegg, et al., 1986, Tet. Let. 27:4051-4051; Froehler, et al., 1986, Nucl. Acid. Res. 14:5399-5407; Garegg, et al., 1986, Tet. Let. 27:4055-4058, Gaffney, et al., 1988), Tet. Let. 29:2619-2622. These chemistries can be performed by a variety of automated oligonucleotide synthesizers available in the market. Alternatively, oligonucleotides can be prepared from existing nucleic acid sequences (e.g., genomic or cDNA) using known techniques, such as those employing restriction enzymes, exonucleases or endonucleases.

For use in vivo, nucleic acids are preferably relatively resistant to degradation (e.g., via endo- and exo-nucleases). Secondary structures, such as stem loops, can stabilize nucleic acids against degradation. Alternatively, nucleic acid stabilization can be accomplished via phosphate backbone modifications. A stabilized nucleic acid can be accomplished via phosphate backbone modifications. A stabilized nucleic acid has at least a partial phosphorothioate modified backbone. Phosphorothioates may be synthesized using automated techniques employing either phosphoramidate or H-phosphonate chemistries. Aryl- and alkyl-phosphonates can be made for example as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., 1990, Chem Rev. 90:544; Goodchild, J., 1990, Bioconjugate Chem. 1:165). 2′-O-methyl nucleic acids with CpG motifs also cause immune activation, as do ethoxy-modified CpG nucleic acids. In fact, no backbone modifications have been found that completely abolish the CpG effect, although it is greatly reduced by replacing the C with a 5-methyl C. DNA N-alkylphosphoramidate linkages may also be of utility in conferring improved stability.

Linking the DNA Nanostructure with the at Least One Targeting Moiety and/or Adjuvant.

Chemistries that can be used to link targeting moieties to the DNA nanostructure are known in the art, such as disulfide linkages, amino linkages, covalent linkages, etc. Additional linkages and modifications can be found on the world-wide-web at trilinkbiotech.com/products/oligo/oligo_modifications.asp.

In certain embodiments, “linked” includes directly linking (covalently or non-covalently binding) the at least one targeting moiety and/or adjuvant to the DNA nanostructure. In certain embodiments, a direct linkage maybe made covalently. For example, the covalent linkage may be made by conjugating the DNA to an amino group on the surface of a peptide using a hetero-cross linker, sulfo SMCC, through Click chemistry, or through the formation of an amide bond. Click chemistry is a process known in the art that uses quantitative chemical reactions of alkyne and azide moieties to create covalent carbon-heteroatom bonds between biochemical species (Rostovtsev, et al., Angew Chem. Int. Ed. Engl., 2002, 41(12): 2596-9). The reaction often uses copper(I) as a catalyst and forms a 1,2,3-triazole between an azide and terminal alkyne (Moses et al., Chem. Soc. Rev. 2007, 36(8):1249-62).

DNA N-alkylphosphoramidates may also be of utility as linkers in certain embodiments.

In certain embodiments, “linked” includes linking the at least one targeting moiety and/or adjuvant to the DNA nanostructure using a linker, e.g., a nucleotide linker, e.g., the nucleotide sequence “AA” or “TT” or “UU”.

In certain embodiments, the linker is a binding pair. In certain embodiments, the “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate, IgG-protein A, antigen-antibody, and the like. In certain embodiments, a first member of the binding pair comprises avidin or streptavidin and a second member of the binding pair comprises biotin.

As used herein the terms “link”, “conjugate” and “engraft” may be used interchangeably.

### Nanovaccines

In certain embodiments, compositions described herein are “nanovaccines”. The term “nanovaccine” refers to a composition capable of producing an immune response. In certain embodiments, the composition of the present invention may be used in the prophylactic or therapeutic treatment of a pathological condition. In certain embodiments, the pathological condition is a disease, for example, HIV or cancer. In certain embodiments, a nanovaccine composition, according to the invention, would produce immunity against disease in individuals. In certain embodiments, the pathological condition is substance abuse or addiction.

In certain embodiments the nanovaccine is about 20-200 nm, such as 50-100 nm in size.

In certain embodiments, the composition further comprises at least one T helper-peptide and at least one adjuvant targeting moieties. In certain embodiments the adjuvant is an oligonucleotide containing at least one immunostimulatory CpG motif.

### Diagnostic & Therapeutic Uses

In the methods of the present invention, the subject may be a vertebrate animal including a human, dog, cat, horse, cow, pig, sheep, goat, chicken, monkey, rat, or mouse.

In one embodiment, the invention provides a method for stimulating an immune response in a subject by administering a therapeutically effective amount of a compound of the invention. This invention provides administering to a subject addicted to nicotine or at risk to become addicted to nicotine, a composition comprising a therapeutically effective dose of a compound of the invention and a pharmaceutically acceptable carrier. “Administering” the pharmaceutical composition of the present invention may be accomplished as described below and by any means known to the skilled artisan.

### Formulations and Methods of Administration

The compositions of the invention may be formulated as pharmaceutical composition and administered to a subject, such as a human patient, in a variety of forms adapted to the chosen route of administration, i.e., orally, mucosally, intranasally, intradermally, intratumorally or parenterally, by intravenous, intramuscular, topical or subcutaneous routes.

Formulations will contain an effective amount of the active ingredient in a vehicle, the effective amount being readily determined by one skilled in the art. “Effective amount” is meant to indicate the quantity of a compound necessary or sufficient to realize a desired biologic effect. For example, an effective amount of a composition described herein could be the amount necessary to eliminate completely or reduce dependency on, or addiction to, nicotine. The active ingredient may typically range from about 1% to about 95% (w/w) of the composition, or even higher or lower if appropriate. The amount for any particular application can vary depending on such factors as the severity of the condition. The quantity to be administered depends upon factors such as the age, weight and physical condition of the animal or the human subject considered for vaccination and kind of concurrent treatment, if any. The quantity also depends upon the capacity of the animal's immune system to synthesize antibodies, and the degree of protection desired. Typically, dosages used in vitro may provide useful guidance in the amounts useful for in situ administration of the composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Reminpton's Pharmaceutical Sciences, 17th ed., Mack Publishing Co., Easton, Pa., 1990, each of which is herein incorporated by reference. Additionally, effective dosages can be readily established by one of ordinary skill in the art through routine trials establishing dose response curves. The subject is immunized by administration of the composition thereof in one or more doses. Multiple doses may be administered as is required to maintain a state of immunity to the target. For example, the initial dose may be followed up with a booster dosage after a period of about four weeks to enhance the immunogenic response. Further booster dosages may also be administered. The composition may be administered multiple (e.g., 2, 3, 4 or 5) times at an interval of, e.g., about 1, 2, 3, 4, 5, 6 or 7, 14, or 21 days apart.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the present compositions may be combined with one or more excipients and used in the form of ingestible tablets, buccal tablets, troches, capsules, elixirs, suspensions, syrups, wafers, and the like. Such preparations should contain at least 0.1% of the present composition. The percentage of the compositions may, of course, be varied and may conveniently be between about 2 to about 60% of the weight of a given unit dosage form. The amount of present composition in such therapeutically useful preparations is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the present composition, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the present compositions may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the present composition that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating a composition described herein in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compositions described herein may be applied in pure form, i.e., when they are liquids. However, it will generally be desirable to administer them to the skin as formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compositions of the present invention can be determined by comparing their in vitro activity, and in vivo activity in animal models. Methods for the extrapolation of effective dosages in mice, and other animals, to humans are known to the art.

The amount of the compositions described herein required for use in treatment will vary with the route of administration and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### GENERAL TERMINOLOGY

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

“Synthetic” aptamers are those prepared by chemical synthesis. The aptamers may also be produced by recombinant nucleic acid methods.

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

By “fragment” or “portion” is meant a full length or less than full length of the nucleotide sequence.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

As noted above, another indication that two nucleic acid sequences are substantially complementary in a Watson-Crick base pairing sense is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched nucleic acid. Specificity is typically the function of post-hybridization washes, the critical factors being the ionic strength and temperature of the final wash solution. For DNA-DNA hybrids, the Tₘ can be approximated from the equation of Meinkoth and Wahl: Tₘ 81.5° C.+16.6 (log M)+0.41 (% GC)−0.61 (% form)−500/L. M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule containing less than 31 sequential nucleotides that is transcribed into an RNAi molecule. Such an isolated RNAi molecule may, for example, form a hairpin structure with a duplex 21 base pairs in length that is complementary or hybridizes to a sequence in a gene of interest, and remains stably bound under stringent conditions (as defined by methods well known in the art, e.g., in Sambrook and Russell, 2001). Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

In certain embodiments a DNA sequence may encode a siRNA, as well as double-stranded interfering RNA molecules, which are also useful to inhibit expression of a target gene.

The terms “protein,” “peptide” and “polypeptide” are used interchangeably herein. As used herein, the terms “a” or “an” are used to mean “one or more.”

“Recombinant DNA molecule” is a combination of DNA sequences that are joined together using recombinant DNA technology and procedures used to join together DNA sequences as described, for example, in Sambrook and Russell (2001).

“Operably-linked” nucleic acids refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The term “amino acid” includes the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in Dextrorotary or Levorotary stereoisomeric forms, as well as unnatural amino acids (e.g., phosphoserine, phosphothreonine, phosphotyrosine, hydroxyproline, and gamma-carboxyglutamate; hippuric acid, octahydroindole-2-carboxylic acid, statine, 1,2,3,4,-tetrahydroisoquinoline-3-carboxylic acid, penicillamine, ornithine, citruline, alpha-methyl-alanine, para-benzoylphenylalanine, phenylglycine, propargylglycine, sarcosine, and tert-butylglycine). The term also comprises natural and unnatural amino acids (Dextrorotary and Levorotary stereoisomers) bearing a conventional amino protecting group (e.g. acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc, and documents cited therein). An amino acid can be linked to the remainder of a compound of formula (I) through the carboxy terminus, the amino terminus, or through any other convenient point of attachment, such as, for example, through the sulfur of cysteine.

The term “peptide” describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound through the carboxy terminus, the amino terminus, or through any other convenient point of attachment, such as, for example, through the sulfur of a cysteine. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right.

The invention will now be illustrated by the following non-limiting Examples.

### Example 1

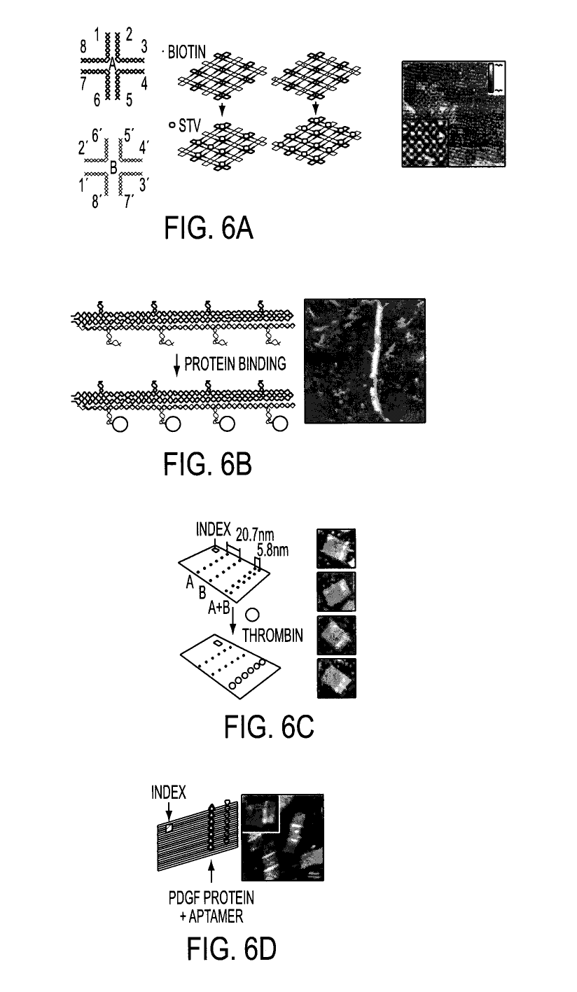

Tetrahedron DNA offers up to 36 sites for engineering various antigenic components. We elected to assemble hapten, Th-epitopes and adjuvants at a ratio of 12:12:12. Specifically, each antigenic component was linked to a unique oligonucleotide and subsequently assembled with the desired ratio to form the DNA-NIC complex. Here, we focused on a single modified NIC for oligonucleotide conjugation in which the linker was attached via the pyrrolidine moiety of NIC. As reported by Janda et al (4), this linker can confer excellent NIC immunogenicity upon conjugation to a protein carrier, to which the NIC-specific antibody generated was used to construct AAV-Nic for the passive immunization (17). The proper display of NIC on the DNA surface is critical to its recognition by NIC-specific B cells. To determine the NIC configuration on the DNA in terms of its ratio and surface presentation, we developed an antibody-based detection method. This method allowed determination whether the NIC-hapten is recognized by the known anti-NIC antibody, which presumably bears the same specificity as the immunoglobulin expressed on the surface of NIC-specific B cells. With anti-NIC antiserum was developed an ELISA-based competition analysis to assess the NIC linkage to DNA. As shown in FIG. 6, we demonstrated that the tetrahedron-assembled NIC shows a much higher level of competition than the monovalent NIColigonucleotide conjugates, at a level approaching that of NICBSA that contains multiple copies of NIC (20-40 on average) which is consistent with the number of NIC-units (i.e., 12) displayed on the DNA. This study gives us the confidence that DNA-directed NIC can effectively present NIC at appropriate configurations to interact with NIC-specific B cells.

### Example 2

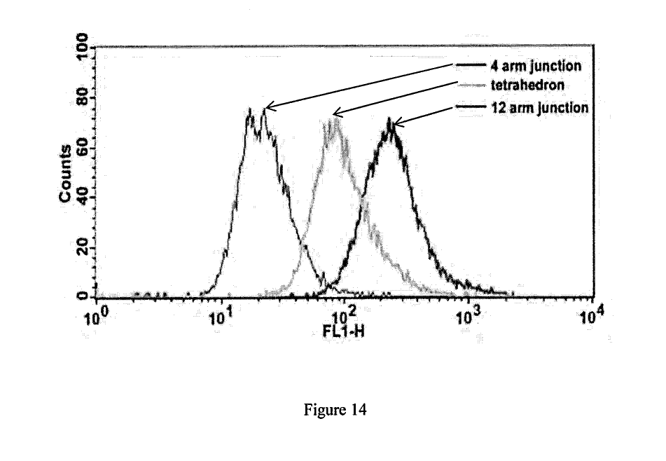

Tetrahedron DNA appears to promote internalization of the model antigen linked to the DNA, more so than a 4-arm branched DNA-structure. This finding seems to be correlated with the immunogenicity presented by the two types of DNA-structures (13). Although the mechanism underlying the internalization profiles of different DNA structures is not clear, it is reasoned that more compact DNA configurations, with more double-stranded DNA (dsDNA), represent a better format for attaching to the cell surface for internalization. To test this idea the tetrahedron DNA was compared to a 12-arm DNA structure that was previously reported by Wang & Seeman (32). The 12-arm DNA structure has essentially the same general structure as the 4-arm branched motif. Yet, the internalization of the 12-arm DNA structure was found higher than the 4-arm branched DNA, even more so than the tetrahedron DNA, as shown in FIG. 14.

### Example 3

Experiments relating to the following are described below: 1) identification of stable DNA-nanoscaffolds for vaccine assembly; 2) evaluation of the immunogenicity of DNA-assembled nicotine haptens; and 3) synthesis of new nicotine haptens for incorporation into either proteins and DNA. As described herein, it has been demonstrated that DNA-scaffolded nicotine vaccines can induce strong anti-nicotine antibody responses, which are effective to reduce nicotine distribution in the brain. In addition to incorporating new nicotine haptens, DNA-nanostructures and immunization regimes have now also been modified and optimized to further enhance nicotine immunogenicity.

1. DNA-Tetrahedron-Nicotine Vaccines: Stable Structures with an Accessible Display of Nicotine Haptens

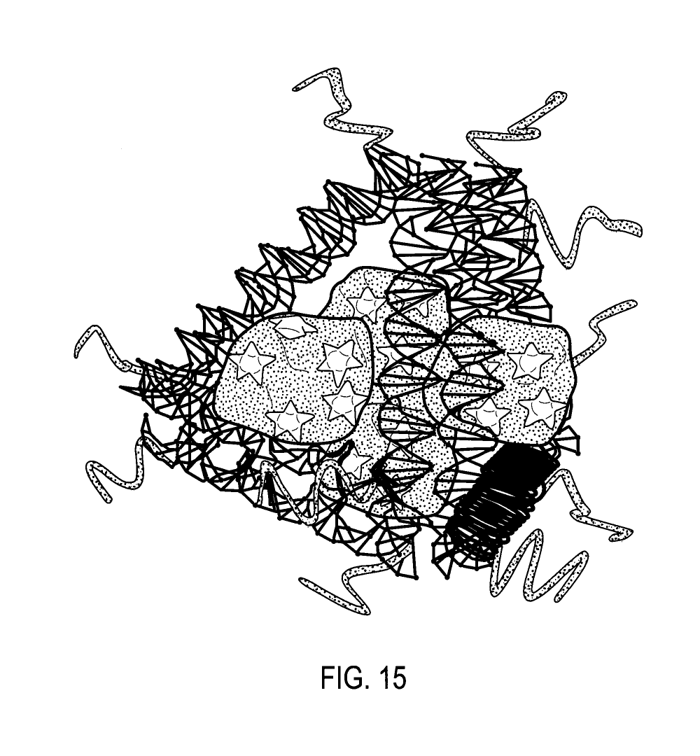

After initial screenings of several DNA-nanostructures, a DNA-tetrahedron was chosen as the assembly platform as it is a simple and stable nano-scale structure with good addressability for attaching nicotine and adjuvants, as shown in FIG. 15. In addition, several unique features of streptavidin (STV) were exploited for the vaccine construction: 1) its binding to biotinylated DNA strands can further stabilize DNA-tetrahedron structures; 2) its conjugation to nicotine afford surface display of multiple nicotine haptens (12-16 per STV tetramer); and 3) its peptides serve as good T helper cell epitopes. Finally, CpG oligonucleotides (CpG-ODN), an TLR9 ligand, are assembled into the DNA-nanostructure through complementary base-paring. Thus, the DNA-nanostructure allows co-delivery of various antigenic components, which is known to be critical for an induction of T-cell dependent B cell responses. This DNA structure also renders good control over the stoichiometry of these components.

### 2. Immunogenicity Assessment of DNA-Based Nicotine Haptens

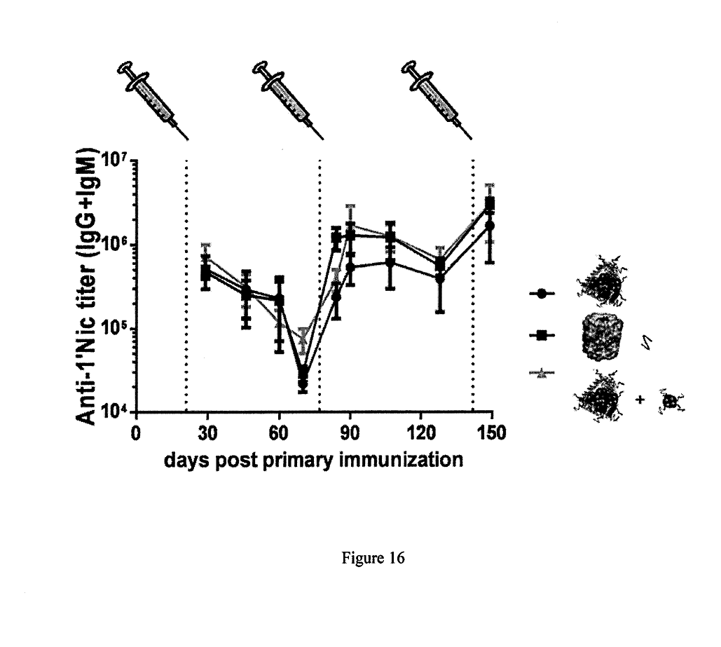

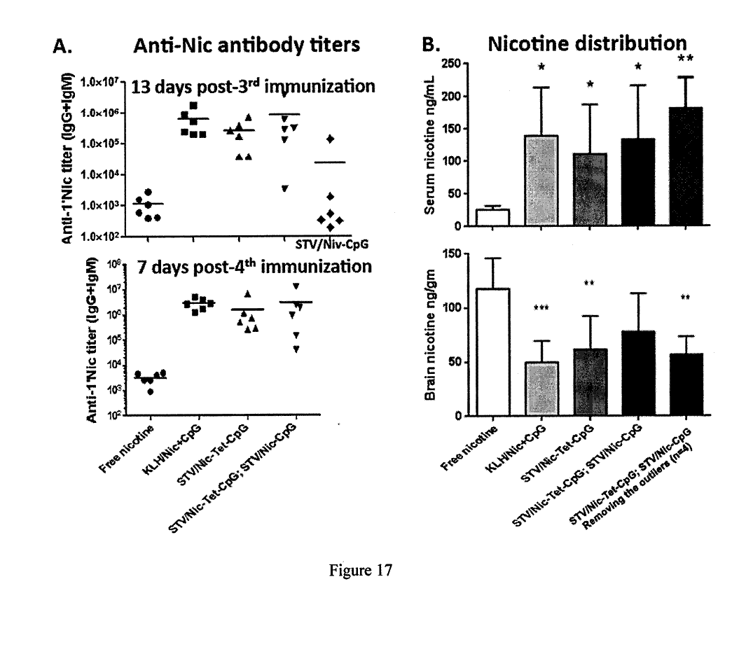

The constructed DNA-based nicotine vaccines were evaluated for their immunogenicity in both C57BL6 and Balb/c mice. With some modifications in immunization regimes, it was found that DNA-scaffolded nicotine vaccines elicited strong anti-nicotine antibody responses, at the level and duration comparable to the one induced by nicotine-KLH conjugates mixed with CpG-ODN (FIG. 16), and also much higher than the one by STV-conjugated nicotine-CpG-ODN (FIG. 17A the top panel), which serves as a control. Interestingly, some mice primed with DNA-scaffolded vaccines followed by boosting with STV-conjugated nicotine-CpG-ODN (referred as priming/boosting) showed a titer (line with triangle, FIG. 16) higher than the one receiving repetitive immunizations with DNA-scaffoded nicotine vaccines (line with circle). This data indicates that immunization regimes may be modified to further improve the immunogenicity.

When these immunized mice were subjected to nicotine pharmacokinetics analysis after 4^{th} immunization via i.p., the mice with high antibody titers (FIG. 17A) demonstrated better nicotine retention in the blood, as shown in FIG. 17B. Thus, the antibody elicited by DNA-assembled nicotine vaccines can sequester a significant amount of nicotine and block its entry into the brain by 50%. It may be noted that 1) repetitive immunization with fully assembled DNA-nanoscaffolded vaccines may compromise the antibody responses; and 2) the group receiving priming/boosting combinations showed variability. In particular, in comparing the antibody titers between 3^{rd} and 4^{th} immunization of priming/boosting region (FIG. 17A), one mouse was found to reduce the antibody level upon 4^{th} immunization. The two mice with low antibody titers showed a very low efficacy in neutralizing nicotine, which were excluded from the data represented in the last column of FIG. 17B.

### 3. Synthesis of New Nicotine Haptens

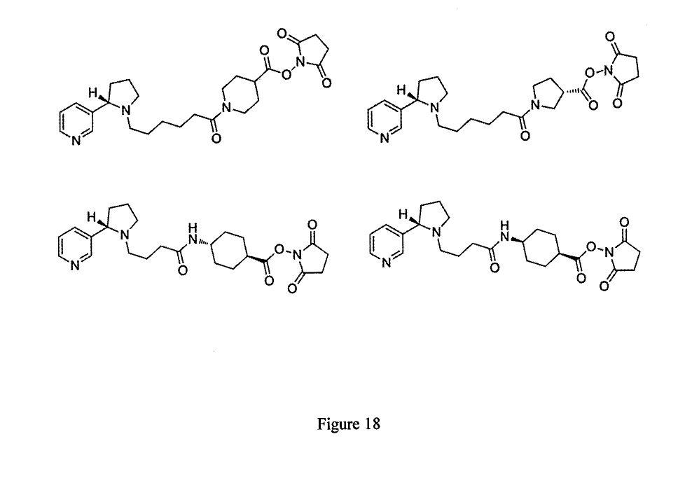

In order to generate efficient antibodies against nicotine, the selection of an optimal linker moiety is important. The length, nature and rigidity of the linker may be considered. Cyclic systems were incorporated along with an internal amide bond to increase the rigidity of the linker; the linker used here was approximately 12-15 Å in length (10-12 atoms), as shown in FIG. 18.

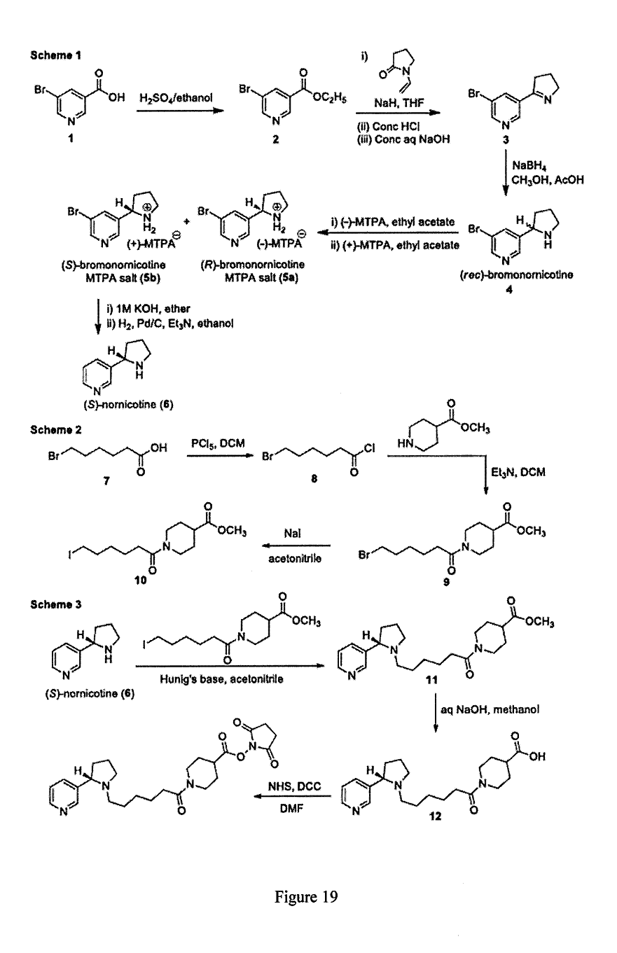

As exemplified for the nicotine hapten shown at the upper left of the FIG. 18, (S)-nornicotine (6) was synthesized, as shown in FIG. 19, starting from 5-bromonicotinic acid (1) (Scheme 1), which upon esterification gave compound 2. Bromo ester 2 was condensed with N-vinyl pyrrolidinone, followed by in situ acid catalyzed hydrolysis, decarboxylation and base-promoted cyclization to afford compound 3. Imine 3 upon reduction with sodium borohydride provided racemic 5-bromonornicotine (4). This racemic mixture was resolved with α-methoxy-α-trifluoromethyl phenyl acetic acid (MTPA) as the corresponding MTPA salts ((R)-5-bromonornicotine MTPA salt (5a) and (S)-5-bromonornicotine MTPA salt (5b)). The (S)-bromonornicotine MTPA salt (5b) upon reductive debromination with hydrogen and palladium catalyst afforded (S)-nornicotine (6, Scheme 1).

The linker was prepared starting from the commercially available 6-bromohexanoic acid (7). This acid was converted into acyl chloride 8 followed by amide bond formation with methyl 4-piperidinecarboxylate, resulting in bromo amide 9. This bromo compound was converted into the corresponding iodide (10, Scheme 2).

(S)-nornicotine (6) was alkylated with iodo compound 10 by using Hunig's base in acetonitrile to afford the nicotine hapten methyl ester (11). This ester upon hydrolysis with base provided the corresponding carboxylic acid (12). This acid was coupled with N-hydroxysuccinimide to give the desired nicotine hapten succinimide ester (Scheme 3).

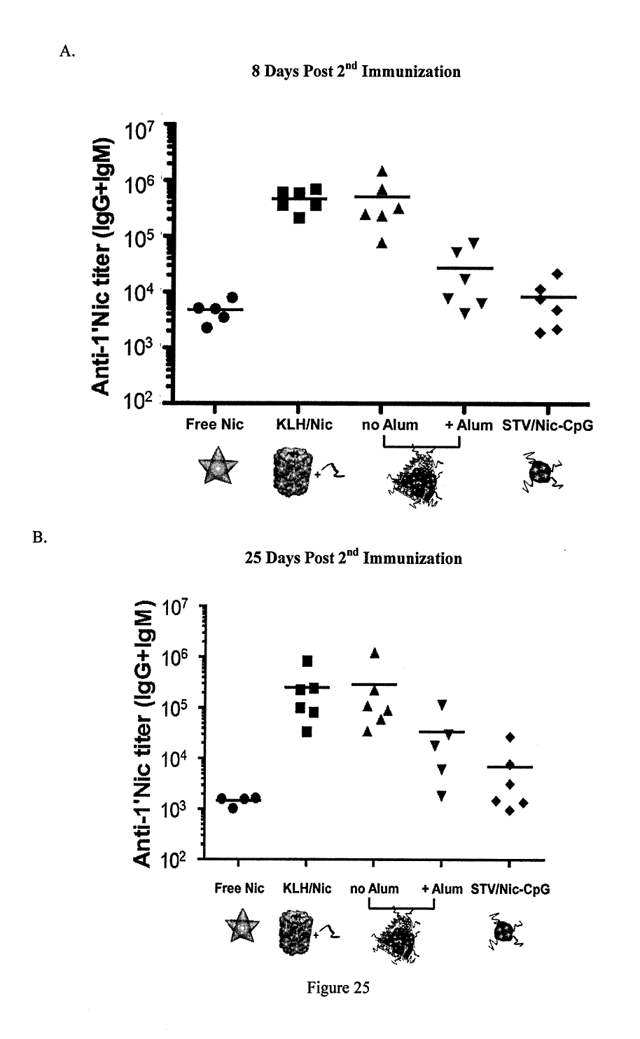

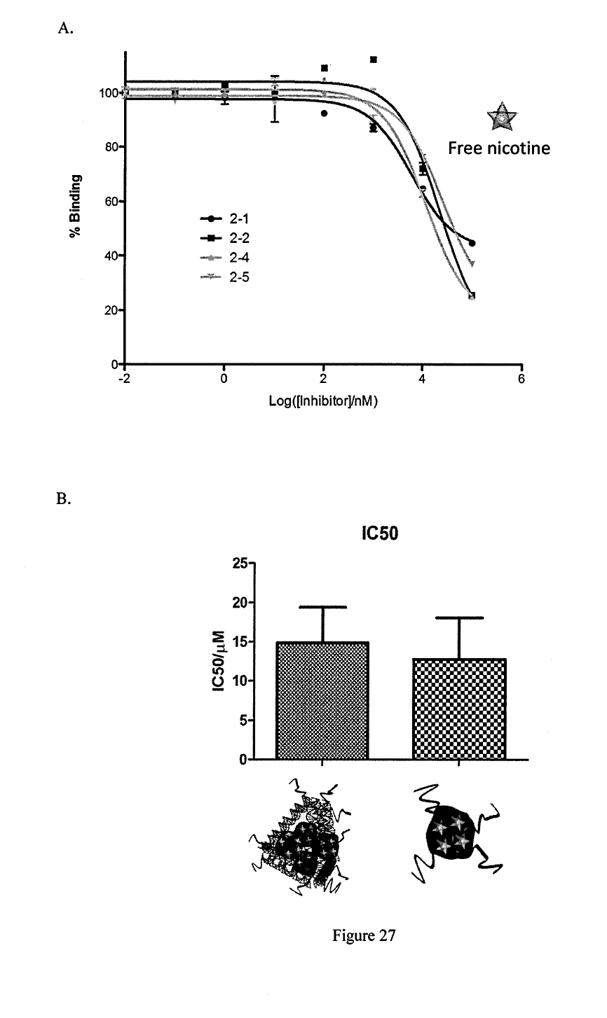

As described herein, the DNA-assembled nicotine vaccines possess a number of advantages: they demonstrate easy and robust self-assembly, show good structural stability (e.g., stable at 37° C.) and properly display antigenic epitopes. Additionally, the vaccines have titers at 10⁵⁻⁶ after only two rounds of immunization (see, e.g., FIG. 25) and the relative affinity is comparable to the reported range (see, e.g., FIG. 27). Finally, no apparent toxicity or anti-DNA autoimmune reactions have been observed.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and “or” and similar referents in the context of describing the invention are to be construed to cover both the singular and the plural, unless otherwise indicated herein or clearly contradicted by context. Thus, for example, reference to “a subject polypeptide” includes a plurality of such polypeptides and reference to “the agent” includes reference to one or more agents and equivalents thereof known to those skilled in the art, and so forth.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventor for carrying out the invention. Variations of those embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

### REFERENCES

1. Maurer, P. and Bachmann, M. F. (2006) Therapeutic vaccines for nicotine dependence. Curr Opin Mol Ther, 8, 11-16.
2. Fahim, R. E., Kessler, P. D., Fuller, S. A. and Kalnik, M. W. (2011) Nicotine vaccines. CNS & neurological disorders drug targets, 10, 905-915.
3. Janda, K. D. and Treweek, J. B. (2012) Vaccines targeting drugs of abuse: is the glass half-empty or half-full? Nat Rev Immunol, 12, 67-72.
4. Isomura, S., Wirsching, P. and Janda, K. D. (2001) An immunotherapeutic program for the treatment of nicotine addiction: hapten design and synthesis. J Org Chem, 66, 4115-4121.
5. Pravetoni, M., Keyler, D. E., Pidaparthi, R. R., Carroll, F. I., Runyon, S. P., Murtaugh, M. P., Earley, C. A. and Pentel, P. R. (2012) Structurally distinct nicotine immunogens elicit antibodies with non-overlapping specificities. Biochemical pharmacology, 83, 543-550.
6. Shlomchik, M. J. and Weisel, F. (2012) Germinal center selection and the development of memory B and plasma cells. Immunol Rev, 247, 52-63.
7. Ingale, S., Wolfert, M. A., Gaekwad, J., Buskas, T. and Boons, G. J. (2007) Robust immune responses elicited by a fully synthetic three-component vaccine. Nat Chem Biol, 3, 663-667.
8. Ghasparian, A., Riedel, T., Koomullil, J., Moehle, K., Gorba, C., Svergun, D. I., Perriman, A. W., Mann, S., Tamborrini, M., Pluschke, G. et al. (2011) Engineered synthetic virus-like particles and their use in vaccine delivery. Chembiochem, 12, 100-109.
9. Jewell, C. M., Lopez, S. C. and Irvine, D. J. (2011) In situ engineering of the lymph node microenvironment via intranodal injection of adjuvant-releasing polymer particles. Proc Natl Acad Sci USA, 108, 15745-15750.
10. George, S. E., Elliott, C. T., McLaughlin, D. P., Delahaut, P., Akagi, T., Akashi, M. and Fodey, T. L. (2012) An investigation into the potential use of nanoparticles as adjuvants for the production of polyclonal antibodies to low molecular weight compounds. Vet Immunol Immunopathol.
11. Moon, J. J., Suh, H., Li, A. V., Ockenhouse, C. F., Yadava, A. and Irvine, D. J. (2012) Enhancing humoral responses to a malaria antigen with nanoparticle vaccines that expand Tfh cells and promote germinal center induction. Proc Natl Acad Sci USA, 109, 1080-1085.
12. Moon, J. J., Suh, H., Polhemus, M. E., Ockenhouse, C. F., Yadava, A. and Irvine, D. J. (2012) Antigendisplaying lipid-enveloped PLGA nanoparticles as delivery agents for a Plasmodium vivax malaria vaccine. PLoS One, 7, e3 1472.
13. Liu, X., Xu, Y., Yu, T., Clifford, C., Liu, Y., Yan, H. and Chang, Y. (2012) A DNA Nanostructure Platform for Directed Assembly of Synthetic Vaccines. Nano Lett.
14. Pentel, P. R., Keyler, D. E. and Kosten, T. R. (2012) Immunotherapies To Treat Drug Addiction. Levine.
15. Shen, X. Y., Orson, F. M. and Kosten, T. R. (2012) Vaccines against drug abuse. Clinical pharmacology & Therapuetics, 91, 61-70.
16. Rioko, S. A., Harris, A. C., LeSage, M. G., Kelyer, D. E. and Pentel, P. R. (2009) Passive immunization with a nicotine-specific monoclonal antibody decreases brain nicotine levels but does not precipitate withdrawl in nicotine-dependent rats. Pharmacol Biochem Behav, 93, 105-111.
17. Hicks, M. J., Rosenberg, J. B., De, B. P., Pagovich, O. E., Young, C. N., Qiu, J. P., Kaminsky, S. M., Hackett, N. R., Worgall, S., Janda, K. D. et al. (2012) AAV-Directed Persistent Expression of a Gene Encoding Anti-Nicotine Antibody for Smoking Cessation. Science translational medicine, 4, 140ra187.
18. Wang, Z., Tapscott, S. J., Chamberlain, J. S. and Storb, R. (2011) Immunity and AAV-Mediated Gene Therapy for Muscular Dystrophies in Large Animal Models and Human Trials. Front Microbiol, 2, 201.
19. Mingozzi, F. and High, K. A. (2012) Immune responses to AAV in clinical trials. Curr Gene Ther, 11, 321-330.
20. Balazs, A. B., Chen, J., Hong, C. M., Rao, D. S., Yang, L. and Baltimore, D. (2012) Antibody-based protection against HIV infection by vectored immunoprophylaxis. Nature, 481, 81-84.
21. Roiko, S. A., Harris, A. C., Keyler, D. E., Lesage, M. G., Zhang, Y. and Pentel, P. R. (2008) Combined active and passive immunization enhances the efficacy of immunotherapy against nicotine in rats. J Pharmacol Exp Ther, 325, 985-993.
22. Moreno, A. Y. and Janda, K. D. (2011) Current challenges for the creation of effective vaccines against drugs of abuse. Expert Rev Vaccines, 10, 1637-1639.
23. Benson, M. J., Erickson, L. D., Gleeson, M. W. and Noelle, R. J. (2007) Affinity of antigen encounter and other early B-cell signals determine B-cell fate. Curr Opin Immunol, 19, 275-280.
24. McHeyzer-Williams, M., Okitsu, S., Wang, N. and McHeyzer-Williams, L. (2011) Molecular programming of B cell memory. Nat Rev Immunol, 12, 24-34.
25. Bettahi, I., Dasgupta, G., Renaudet, O., Chentoufi, A. A., Zhang, X., Carpenter, D., Yoon, S., Dumy, P. and BenMohamed, L. (2009) Antitumor activity of a self-adjuvanting glyco-lipopeptide vaccine bearing B cell, CD4+ and CD8+T cell epitopes. Cancer Immunol Immunother, 58, 187-200.
26. Schellenberger, M. T., Grova, N., Farinelle, S., Willieme, S., Revets, D. and Muller, C. P. (2012) Immunogenicity of a promiscuous T cell epitope peptide based conjugate vaccine against benzo[a]pyrene: redirecting antibodies to the hapten. PLoS One, 7, e38329.
27. Bourquin, C., D., A., Zwiorek, K., Lanz, A.-L., Fuchs, S., Weigel, S., Wurzenberger, C., von der Borch, P., Golic, M., S., M. et al. (2008) Targeting CpG oligonucleotides to the lymph node by nanoparticles elicits efficient antitumoral immunity. J. Immunol., 181, 2990-2998.
28. Kishimoto, K., Altreuter, D., Johnstong, L., Keller, P. and Pittet, L. (2012), Society for Reearch o Nicotine and Tobacco, Houston, Tex., pp. 43.
29. Seeman, N. C. (2010) Structural DNA nanotechnology: growing along with Nano Letters. Nano Lett, 10, 1971-1978.
30. Han, D., Pal, S., Nangreave, J., Deng, Z., Liu, Y. and Yan, H. (2011) DNA origami with complex curvatures in three-dimensional space. Science, 332, 342-346.
31. Liu, X., Yan, H., Liu, Y. and Chang, Y. (2011) Targeted cell-cell interactions by DNA nanoscaffoldtemplated multivalent bispecific aptamers. Small, 7, 1673-1682.
32. Wang, X. and Seeman, N. C. (2007) Assembly and characterization of 8-arm and 12-arm DNA branched junctions. J Am Chem Soc, 129, 8169-8176.
33. Bishop, G. A., Ramirez, L. M., Baccam, M., Busch, L. K., Pederson, L. K. and Tomai, M. A. (2001) The immune response modifier resiquimod mimics CD40-induced B cell activation. Cell Immunol, 208, 9-17.
34. Hanten, J. A., Vasilakos, J. P., Riter, C. L., Neys, L., Lipson, K. E., Alkan, S. S. and Birmachu, W. (2008) Comparison of human B cell activation by TLR7 and TLR9 agonists. BMC Immunol, 9, 39.
35. Duthie, M. S., Windish, H. P., Fox, C. B. and Reed, S. G. (2011) Use of defined TLR ligands as adjuvants within human vaccines. Immunol Rev, 239, 178-196.
36. El-Sagheer, A. H. and Brown, T. (2010) Click chemistry with DNA. Chem Soc Rev, 39, 1388-1405.
37. Tomai, M. A., Imbertson, L. M., Stanczak, T. L., Tygrett, L. T. and Waldschmidt, T. J. (2000) The immune response modifiers imiquimod and R-848 are potent activators of B lymphocytes. Cell Immunol, 203, 55-65.
38. Gerster, J. F., Lindstrom, K. J., Miller, R. L., Tomai, M. A., Birmachu, W., Bomersine, S. N., Gibson, S. J., Imbertson, L. M., Jacobson, J. R., Knafla, R. T. et al. (2005) Synthesis and structure-activity-relationships of 1H-imidazo[4,5-c]quinolines that induce interferon production. J Med Chem, 48, 3481-3491.
39. Pentel, P. R., Malin, D. H., Ennifar, S., Hieda, Y., Keyler, D. E., Lake, J. R., Milstein, J. R., Basham, L. E., Coy, R. T., Moon, J. W. et al. (2000) A nicotine conjugate vaccine reduces nicotine distribution to brain and attenuates its behavioral and cardiovascular effects in rats. Pharmacol Biochem Behav, 65, 191-198.
40. Satoskar, S. D., Keyler, D. E., LeSage, M. G., Raphael, D. E., Ross, C. A. and Pentel, P. R. (2003) Tissuedependent effects of immunization with a nicotine conjugate vaccine on the distribution of nicotine in rats. Int Immunopharmacol, 3, 957-970.
41. LeSage, M. G., Keyler, D. E., Hieda, Y., Collins, G., Burroughs, D., Le, C. and Pentel, P. R. (2006) Effects of a nicotine conjugate vaccine on the acquisition and maintenance of nicotine selfadministration in rats. Psychopharmacology (Berl), 184, 409-416.
42. Wee, S., Hicks, M. J., De, B. P., Rosenberg, J. B., Moreno, A. Y., Kaminsky, S. M., Janda, K. D., Crystal, R. G. and Koob, G. F. (2012) Novel cocaine vaccine linked to a disrupted adenovirus gene transfer vector blocks cocaine psychostimulant and reinforcing effects. Neuropsychopharmacology: official publication of the American College of Neuropsychopharmacology, 37, 1083-1091.
43. Maurer, P., Jennings, G. T., Willers, J., Rohner, F., Lindman, Y., Roubicek, K., Renner, W. A., Muller, P. and Bachmann, M. F. (2005) A therapeutic vaccine for nicotine dependence: preclinical efficacy, and phase I safety and immunogenicity. Eur J Immunol, 35, 2031-2040.
44. Ke, Y., Lindsay, S., Chang, Y., Liu, Y. and Yan, H. (2008) Self-assembled water-soluble nucleic acid probe tiles for label-free RNA hybridization assays. Science, 319, 180-183.
45. Alexander, J., de Guerci, M.-F., Maewal, A., Qiao, L., Fikes, J., Chesnut, R. W., Paulson, J., Bundle, D. R., DeFrees, S. and Sette, A. (2000) Linear PADRE T Helper Epitope and Carbohydrate B Cell Epitope Conjugates Induce Specific High Titer IgG Antibody Responses. J. Immunol., 164; 1625-1633.
46. Wang, P., Sidney, J., Dow, C., Mothe, B., Sette, A. and Peters, B. (2008) A systematic assessment of MHC class II peptide binding predictions and evaluation of a consensus approach. PLoS Comput Biol, 4, e1000048.
47. Zhang, H., Wang, P., Papangelopoulos, N., Xu, Y., Sette, A., Bourne, P. E., Lund, O., Ponomarenko, J., Nielsen, M. and Peters, B. (2010) Limitations of Ab initio predictions of peptide binding to MHC class II molecules. PLoS One, 5, e9272.
48. Kim, Y., Ponomarenko, J., Zhu, Z., Tamang, D., Wang, P., Greenbaum, J., Lundegaard, C., Sette, A., Lund, O., Bourne, P. E. et al. (2012) Immune epitope database analysis resource. Nucleic Acids Res, 40, W525-530.
49. Moreno, A. Y. and Janda, K. D. (2009) Immunopharmacotherapy: vaccination strategies as a treatment for drug abuse and dependence. Pharmacol Biochem Behav, 92, 199-205.
50. Bergstrom, D. E., Ruth, J. and Warwick, P. (1981)C-5-Substituted pyrimidine nucleosides. 3. Reaction of allylic chlorides, alcohols, and acetates with pyrimidine nucleoside derived organopalladium intermediates. J. Org. Chem., 46, 1432-1441.
51. LeSage, M. G., Keyler, D. E. and Pentel, P. R. (2006) Current status of immunologic approaches to treating tobacco dependence: vaccines and nicotine-specific antibodies. AAPS J, 8, E65-75.
52. Cornuz, J., Zwahlen, S., Jungi, W. F., Osterwalder, J., Klingler, K., van Melle, G., Bangala, Y., Guessous, I., Muller, P., Willers, J. et al. (2008) A vaccine against nicotine for smoking cessation: a randomized controlled trial. PLoS ONE, 3, e2547.
53. Lindblom, N., de Villiers, S. H., Semenova, S., Kalayanov, G., Gordon, S., Schilstrom, B., Johansson, A. M., Markou, A. and Svensson, T. H. (2005) Active immunisation against nicotine blocks the reward facilitating effects of nicotine and partially prevents nicotine withdrawal in the rat as measured by dopamine output in the nucleus accumbens, brain reward thresholds and somatic signs. Naunyn Schmiedebergs Arch Pharmacol, 372, 182-194.
54. de Villiers, S. H., Lindblom, N., Kalayanov, G., Gordon, S., Baraznenok, I., Malmerfelt, A., Marcus, M. M., Johansson, A. M. and Svensson, T. H. (2010) Nicotine hapten structure, antibody selectivity and effect relationships: results from a nicotine vaccine screening procedure. Vaccine, 28, 2161-2168.
55. Hatsukami, D. K., Jorenby, D. E., Gonzales, D., Rigotti, N. A., Glover, E. D., Oncken, C. A., Tashkin, D. P., Reus, V. I., Akhavain, R. C., Fahim, R. E. et al. (2011) Immunogenicity and smoking-cessation outcomes for a novel nicotine immunotherapeutic. Clinical pharmacology and therapeutics, 89, 392-399.
56. Hieda, Y., Keyler, D. E., Ennifar, S., Fattom, A. and Pentel, P. R. (2000) Vaccination against nicotine during continued nicotine administration in rats: immunogenicity of the vaccine and effects on nicotine distribution to brain. Int J Immunopharmacol, 22, 809-819.
57. Keyler, D. E., Dufek, M. B., Calvin, A. D., Bramwell, T. J., LeSage, M. G., Raphael, D. E., Ross, C. A., Le, C. T. and Pentel, P. R. (2005) Reduced nicotine distribution from mother to fetal brain in rats vaccinated against nicotine: time course and influence of nicotine dosing regimen. Biochem Pharmacol, 69, 1385-1395.
58. Keyler, D. E., Roiko, S. A., Benlhabib, E., LeSage, M. G., St Peter, J. V., Stewart, S., Fuller, S., Le, C. T. and Pentel, P. R. (2005) Monoclonal nicotine-specific antibodies reduce nicotine distribution to brain in rats: dose- and affinity-response relationships. Drug Metab Dispos, 33, 1056-1061.
59. Pravetoni, M., Keyler, D. E., Raleigh, M. D., Harris, A. C., Lesage, M. G., Mattson, C. K., Pettersson, S. and Pentel, P. R. (2011) Vaccination against nicotine alters the distribution of nicotine delivered via cigarette smoke inhalation to rats. Biochemical pharmacology, 81, 1164-1170.
60. Keyler, D. E., Roiko, S. A., Earley, C. A., Murtaugh, M. P. and Pentel, P. R. (2008) Enhanced immunogenicity of a bivalent nicotine vaccine. Int Immunopharmacol, 8, 1589-1594.
61. Pentel, P. R., Dufek, M. B., Roiko, S. A., Lesage, M. G. and Keyler, D. E. (2006) Differential effects of passive immunization with nicotine-specific antibodies on the acute and chronic distribution of nicotine to brain in rats. J Pharmacol Exp Ther, 317, 660-666.
62. Lerman, C., LeSage, M. G., Perkins, K. A., O'Malley, S. S., Siegel, S. J., Benowitz, N. L. and Corrigall, W. A. (2007) Translational research in medication development for nicotine dependence. Nat Rev Drug Discov, 6, 746-762.
63. LeSage, M. G., Keyler, D. E., Shoeman, D., Raphael, D., Collins, G. and Pentel, P. R. (2002) Continuous nicotine infusion reduces nicotine self-administration in rats with 23-h/day access to nicotine. Pharmacol Biochem Behav, 72, 279-289.
64. LeSage, M. G., Keyler, D. E., Collins, G. and Pentel, P. R. (2003) Effects of continuous nicotine infusion on nicotine self-administration in rats: relationship between continuously infused and self-administered nicotine doses and serum concentrations. Psychopharmacology (Berl), 170, 278-286.
65. Jacob, P., Wilson, M. and Benowitz, N. L. (1981) Improved gas chromatographic method for the determination of nicotine and cotinine in biologic fluids. J Chromatog, 222, 61-70.

What is claimed is:

1. A compound comprising a DNA-nanostructure and at least one targeting moiety,
wherein the at least one targeting moiety is linked to the DNA-nanostructure; and
wherein the at least one targeting moiety is nicotine or an analogue thereof.

2. The compound of claim 1, wherein the nicotine moiety is

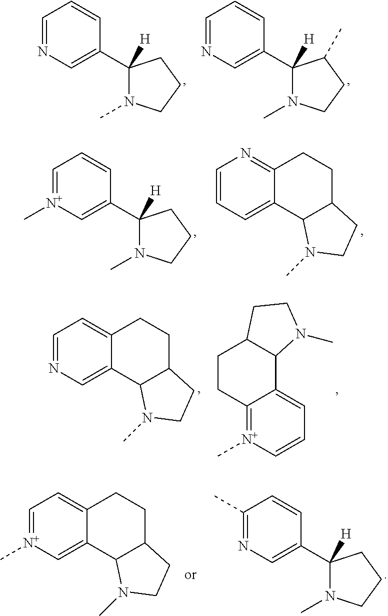

3. The compound of claim 2, wherein the nicotine moiety is

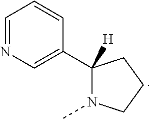

4. The compound of claim 1, wherein the nicotine moiety is conjugated to the 5-position of a thymine nucleobase of the DNA-nanostructure.

5. The compound of claim 1, wherein the nicotine moiety is 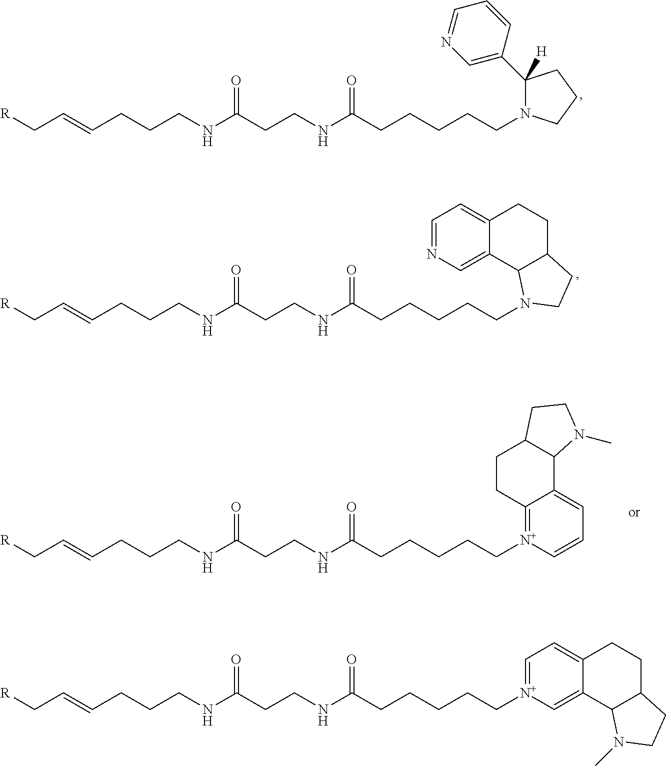
wherein R is the 5-position of a thymine nucleobase in the DNA-nanostructure.

6. The compound of claim 1, wherein the DNA-nanostructure is a DNA-tetrahedron.

7. The compound of claim 1, wherein the DNA-nanostructure is a DNA-branch.

8. The compound of claim 7, wherein the DNA-branch comprises four, eight or twelve oligonucleotides.

9. The compound of claim 1, wherein one oligonucleotide contains at least one CpG motif.

10. The compound of claim 1, wherein at least one targeting moiety is an adjuvant, wherein the adjuvant is linked to the DNA nanostructure.

11. The compound of claim 10, wherein the at least one adjuvant is an oligonucleotide containing at least one immunostimulatory CpG motif.

12. The compound of claim 11, wherein the oligonucleotide is from about 8-30 bases in length.

13. The compound of claim 1, wherein the DNA-nanostructure comprises at least two targeting moieties that are independently the same or different.

14. The compound of claim 13, wherein the targeting moieties are the same.

15. The compound of claim 13, wherein the targeting moieties are different.

16. The compound of claim 1, wherein at least one targeting moiety is a T-helper peptide and at least one targeting moiety is an adjuvant linked to the DNA nanostructure.

17. The compound of claim 16, wherein the adjuvant is an oligonucleotide containing at least one CpG motif.

18. A composition comprising a compound of claim 1, in combination with a physiologically-acceptable, non-toxic vehicle.

19. A method of inducing an immune response in a subject, comprising administering to the subject a therapeutically effective amount of a compound according to claim 1.

20. A method of inducing the production of high affinity neutralizing antibodies or inhibitory antibodies comprising administering a compound of claim 1 to a subject.
